(19) United States
(12) Patent Application Publication
(10) Pub. No.: US 2011/0313132 A1
(43) Pub. Date: Dec. 22, 2011

(54) PROCESS FOR THE PRODUCTION OF PREFORMED CONJUGATES OF ALBUMIN AND A THERAPEUTIC AGENT

(76) Inventors: Dominique P. BRIDON, San Francisco, CA (US); Nathalie BOUSQUET-GAGNON, Lachine (CA); Omar QURAISHI, Westmount (CA)

(21) Appl. No.: 13/047,702

(22) Filed: Mar. 14, 2011

Related U.S. Application Data
(63) Continuation of application No. 11/645,297, filed on Dec. 22, 2006, now abandoned.
(60) Provisional application No. 60/753,680, filed on Dec. 22, 2005.

(51) Int. Cl. C07K 14/76; C07K 19/00; C07K 1/20

(52) U.S. Cl. 530/358; 530/369

(57) ABSTRACT

The present invention provides processes for the production of preformed albumin conjugates. In particular, the invention provides processes for the in-vitro conjugation of a therapeutic compound to recombinant albumin, wherein a therapeutic compound comprising a reactive group is contacted to recombinant albumin in solution to form a conjugate. The processes provide for conjugation to albumin species of increasing homogeneity. The resulting conjugate is purified by chromatography, in particular hydrophobic interaction chromatography comprising phenyl sepharose and butyl sepharose chromatography.

This application is a continuation of U.S. patent application Ser. No. 11/645,297, filed on Dec. 22, 2006, which claims benefit of priority of U.S. provisional application No. 60/753,680, filed on Dec. 22, 2005, the contents of all are hereby incorporated by reference in their entireties.

### 1. FIELD OF THE INVENTION

The present invention provides processes for the production of preformed albumin conjugates. In particular, the invention provides processes for the in-vitro conjugation of a therapeutic compound to recombinant albumin, wherein a therapeutic compound comprising a reactive group is contacted to recombinant albumin in solution to form a conjugate.

### 2. BACKGROUND OF THE INVENTION

Therapeutic molecules must meet rigorous standards in order to be used in humans. In addition to being safe and effective, they must be available in sufficient amounts for sufficient time in the human body to be effective. Unfortunately, many proposed therapeutic molecules are either cleared or degraded, or both, from the human body thereby limiting their effectiveness for treatment. Many proposed peptide therapeutics suffer from such deficiencies in pharmacokinetics.

Breakthroughs have been achieved in the pharmacokinetics of some proposed therapeutics by covalently linking them to carrier molecules such as albumin. Indeed, several albumin conjugates are in clinical trials in humans.

Thus, efficient and effective methods are needed for the production and purification of such albumin conjugates.

### 3. SUMMARY OF THE INVENTION

The present invention provides processes for the production of preformed conjugates of albumin. In certain aspects, this invention provides processes for producing albumin in a host cell, contacting the albumin with a compound which comprises a therapeutic group and a reactive group, under conditions wherein a covalent bond can be formed between the reactive group and cysteine 34 of albumin, and purifying the resulting conjugate formed thereby.

In one aspect, the present invention provides a process for the production of preformed conjugates of albumin, the process comprising the steps of producing albumin in a host cell; partially purifying the albumin product to reduce host proteins, antigens, endotoxins, and the like; contacting the albumin with a compound under conditions that facilitate conjugation between cysteine 34 of albumin and the reactive group of the compound; and purifying the resulting conjugate by one or more hydrophobic interaction chromatography steps, optionally followed by ultrafiltration and formulation.

Thus, one embodiment of the invention provides a process for producing preformed conjugates of albumin, comprising the steps of:
(a) producing recombinant albumin in a host cell;
(b) purifying recombinant albumin from the host cell;
(c) contacting the purified recombinant albumin with a compound, said compound comprising a reactive group, under reaction conditions wherein the reactive group is capable of covalently binding the Cys34 thiol of recombinant albumin to form a conjugate; and
(d) purifying the conjugate by hydrophobic interaction chromatography, optionally followed by ultrafiltration and formulation.

In certain embodiments, the process further comprises enrichment of mercaptalbumin, i.e. albumin composed of free and reactive cysteine 34, prior to the conjugation reaction of step (c). While not intending to be bound by any particular theory of operation, it is believed that oxidation, or “capping” of the cysteine 34 thiol of albumin by cysteine, glutathione, metal ions, or other adducts can reduce the specificity of conjugation to the reactive group of the compound. Accordingly, mercaptalbumin can be enriched from heterogeneous pools of reduced and oxidized albumin by contact with agents known in the art to be capable of converting capped albumin-Cys³⁴ to albumin-Cys³⁴-SH. In certain embodiments, the mercaptalbumin can be enriched by contacting the albumin with thioglycolic acid (TGA). In certain embodiments, the mercaptalbumin can be enriched by contacting the albumin with dithiothreitol (DTT). In some embodiments, mercaptalbumin may be enriched by subjecting the albumin to hydrophobic interaction chromatography, using phenyl or butyl sepharose, or a combination thereof. In other embodiments, mercaptalbumin may be enriched by contacting the albumin with TGA or DTT, followed by purification by hydrophobic interaction chromatography, using phenyl or butyl sepharose resin, or both.

In certain embodiments, the process further comprises reduction of glycated albumin prior to the conjugation reaction of step (c). Reduction of non-enzymatically glycated forms of albumin may be carried out by any technique known to those of skill in the art for reducing glycated albumin. In some embodiments, non-enzymatically glycated albumin may be reduced from the albumin solution by subjecting the solution to affinity chromatography, for instance using aminophenylboronic acid agarose resin, or concanavalin A sepharose, or a combination thereof.

A second aspect of the invention provides a process for the production of preformed conjugates of albumin, wherein recombinant albumin produced by a host cell in a liquid medium is contacted with a compound to form the conjugate, without intervening purification of the recombinant albumin from the culture medium. Thus, embodiments of the invention provides processes for producing preformed conjugates of albumin, the processes comprising the steps of:
(a) producing recombinant albumin in a host cell, wherein the host cell is cultured in a liquid medium;
(b) contacting the liquid medium with a compound, said compound comprising a reactive group, under reaction conditions wherein the reactive group is capable of covalently binding the Cys34 thiol of recombinant albumin contained therein to form a conjugate; and
(c) purifying the conjugate by hydrophobic interaction chromatography optionally followed by ultrafiltration and formulation.

In certain embodiments, the processes further comprise the step of lysing the host cell prior to the conjugation reaction of step (b) to facilitate release of intracellularly stored albumin. In certain embodiments, the processes further comprise the step of separating the host cell, whether intact or lysed, from the liquid medium, thus providing a crude supernatant for the conjugation reaction of step (b).

Any recombinant albumin known to those of skill in the art may be used to form a conjugate according to the processes of the invention. In some embodiments, the recombinant albumin is mammalian albumin, such as, for instance, mouse, rat, bovine, ovine, or human albumin. In a preferred embodiment, the albumin is human recombinant albumin. In some embodiments, the albumin is a fragment, variant, or derivative of human recombinant albumin. In some embodiments, the albumin is an albumin derivative comprising recombinant albumin genetically fused to a therapeutic peptide.

Further, any therapeutic compound known to those of skill in the art may be used to form a conjugate according to the processes of the present invention. In some embodiments, the therapeutic moiety of the compound is selected from the group consisting of a peptide, a protein, an organic molecule, RNA, DNA, and a combination thereof. In some embodiments, the compound comprises a therapeutic peptide, or a derivative thereof, having a molecular weight of less than 30 kDa. Exemplary therapeutic peptides include insulinotropic peptides such as glucacon-like peptide 1 (GLP-1), exendin-3 and exendin-4; and growth hormone releasing factor (GRF). In a particular embodiment, the therapeutic moiety is glucagon-like peptide 1, or a derivative thereof. In a particular embodiment, the therapeutic moiety of the compound is exendin-3, or a derivative thereof. In a particular embodiment, the therapeutic moiety of the compound is exendin-4, or a derivative thereof. In a particular embodiment, the therapeutic moiety is human GRF, or a derivative thereof.

In certain embodiments, the compound comprises a reactive group attached to the therapeutic moiety, either directly or via a linking group. In some embodiments, the reactive group is a Michael acceptor, a succinimidyl-containing group, a maleimido-containing group, or an electrophilic acceptor. In some embodiments, the reactive group is a chemical moiety capable of disulfide exchange. In some embodiments, the reactive group comprises a free thiol. In certain embodiments, the reactive group is a cysteine residue. Linking groups for indirect attachment of the reactive group include, but are not limited to, (2-amino)ethoxy acetic acid (AEA), ethylenediamine (EDA), and 2-[2-(2-amino)ethoxy)]ethoxy acetic acid (AEEA). Where the therapeutic moiety is a peptide, the reactive group may be attached to any residue of the peptide. Useful sites of attachment include the amino terminus, the carboxy terminus, and amino acid side chains.

In accordance with certain processes of the present invention, recombinant albumin is produced in a host cell. Any host cell capable of producing an exogenous recombinant protein may be useful for the processes described herein. In some embodiments, the host cell can be a yeast, bacteria, plant, insect, animal, or human cell transformed to produce recombinant albumin. In some embodiments, the host is cultured in a liquid medium. In certain embodiments the host can be a bacteria strain, for example Escherichia coli and Bacillus subtilis. In other embodiments, the host can be a yeast strain, for example Saccharomyces cerevisiae, Pichia pastoris, Kluyveromyces lactis, Arxula adeninivorans, and Hansenula polymorpha. In a particular embodiment, the host is Pichia pastoris.

In further accordance with the processes of the invention, a crude or partially purified recombinant albumin solution is contacted with a compound comprising a reactive group, under reaction conditions wherein the reactive group is capable of covalently binding the recombinant albumin to form a conjugate. In some embodiments, the reactions conditions comprise a reaction temperature between 1-37° C., or more preferably between 20-25° C. In certain embodiments, the recombinant albumin is contacted with the compound in a solution comprising a low to neutral pH. In some embodiments, the pH is between about 4.0 and 7.0. In certain embodiments, the recombinant albumin is contacted with the compound by dropwise addition of the compound over a period of at least 30 minutes. In some embodiments, the final molar ratio of the compound to recombinant albumin is between 0.1:1 and 1:1. In some embodiments, the final molar ratio of the compound to recombinant albumin is between 0.5:1 and 0.9:1. In a particular embodiment, the final molar ratio of the compound to recombinant albumin is about 0.7:1.

In further accordance with the processes of the invention, the conjugate is purified by hydrophobic interaction chromatography (HIC). In one embodiment, a first purification step comprises subjecting the conjugation reaction to phenyl sepharose chromatography. In certain embodiments, this step separates non-conjugated compound from albumin species, whether free or conjugated. In certain embodiments, the phenyl sepharose column is equilibrated in a buffer having relatively low salt content and neutral pH, e.g., a phosphate buffer of pH 7.0 comprising 5 mM sodium octanoate and 5 mM ammonium sulfate. Under these conditions, non-conjugated compound is capable of binding to the resin while the conjugate is capable of flowing through the column.

In certain embodiments, purification of the conjugate further comprises a mild degradation step following phenyl sepharose chromatography to reduce or destabilize any side reaction products comprising non-Cys34 albumin conjugates. The degradation may be accomplished by incubating the phenyl sepharose flow-through at room temperature for up to 7 days before proceeding further with purification. In certain embodiments, the mild degradation step is followed by a second application to phenyl sepharose to further separate degradation products, i.e., non-conjugated compound from the conjugate.

In certain embodiments, purification of the conjugate further comprises a second HIC step wherein the phenyl sepharose flow-through is subjected to butyl sepharose chromatography to further isolate the conjugate from non-conjugated albumin, dimeric non-conjugated albumin, and residual non-conjugated compound. In certain embodiments, the butyl sepharose column is equilibrated in a buffer at or near neutral pH comprising 5 mM sodium octanoate and 750 mM ammonium sulfate. In certain embodiments, where the molecular weight of the compound is relatively low, e.g., 2 kDa or less, the salt conditions and gradient may be altered. For instance, a starting ammonium sulfate concentration of 1.5 M may be chosen. In certain embodiments, elution may be achieved using either a linear or stepwise decreasing salt gradient, or a combination thereof, wherein non-conjugated albumin is eluted with 750 mM ammonium sulfate, dimeric non-conjugated albumin is eluted with 550 mM ammonium sulfate, compound-albumin conjugates is eluted with 100 mM ammonium sulfate, and unconjugated compound and other species are eluted with water. These species may include, for example, dimeric, trimeric, or polymeric albumin conjugates, or albumin conjugate products comprising a stoichiometry of compound to albumin greater than 1:1.

In certain embodiments, purification of the conjugate further comprises washing and concentrating the conjugate by ultrafiltration following HIC. In some embodiments, sterile water, saline, or buffer may be used to remove ammonium sulfate and buffer components from the purified conjugate.

### 4. BRIEF DESCRIPTION OF THE DRAWINGS

FIG. 1 presents DEAE Sepharose anion exchange purification of recombinant human albumin expressed from Pichia pastoris. Recombinant human albumin elutes in Fraction 2.

FIG. 2 presents Q Sepharose anion exchange purification of recombinant human albumin expressed from Pichia pastoris. Recombinant human albumin elutes in Fraction 2.

FIG. 3 presents HiTrap™ Blue affinity purification of recombinant human albumin expressed from Pichia pastoris. Recombinant human albumin elutes in Fraction 2.

FIG. 4 presents phenyl sepharose hydrophobic interaction purification of recombinant human albumin expressed from Pichia pastoris. Recombinant human albumin elutes in Fraction 2 and 3.

FIG. 5 presents phenyl sepharose hydrophobic interaction purification of recombinant human albumin expressed from Pichia pastoris and treated with thioglycolate for enrichment of mercaptalbumin. Recombinant mercaptalbumin elutes in Fraction 2.

FIG. 6 presents Amino-Phenyl Boronic Acid affinity chromatography of human serum albumin for the reduction of non-enzymatically glycated albumin species, particularly those composed of glucose. Non-glycated albumin species do not bind to the resin (Fraction 1), whereas the presence of glycated forms of albumin may be isolated following their elution from the resin (Fraction 2).

FIG. 7 presents Concanavalin A (Con A) affinity chromatography of human serum albumin for the separation of non-glycated albumin species (Fraction 1) from non-enzymatically glycated albumin species, particularly those composed of sugars other than glucose such as mannose, galactose, lactose, and the like (Fraction 2).

FIG. 8 presents an HPLC chromatogram of unbound DAC-Exendin-4 found post-conjugation between DAC-Exendin-4 CJC-1134 and rHA prior to loading onto Phenyl-Sepharose flow-through column. Retention time of unbound CJC-1134 is 8.2 min, and that of the albumin conjugate is after 12 min.

FIG. 9 presents phenyl sepharose hydrophobic interaction chromatography of a conjugation reaction between DAC-Exendin-4 (CJC-1134) and recombinant human albumin. Phenyl-Sepharose was pre-equilibrated in 20 mM sodium phosphate buffer (pH 7.0) composed of 5 mM sodium octanoate and 5 mM ammonium sulfate. Direct loading of conjugation reaction onto this resin enables physical separation of protein (albumin and conjugated albumin) observed in flow-through from unbound DAC-Exendin-4 (CJC-1134). Therefore, capacity of this resin is reserved primarily for unbound compound composed of a reactive moiety.

FIG. 10 presents an HPLC chromatogram of unbound DAC-Exendin-4 found post-conjugation between DAC-Exendin-4 (CJC-1134) and rHA following loading of reaction mixture onto Phenyl-Sepharose flow-through column. Retention time of unbound CJC-1134 is 8.2 min. and that of the albumin conjugate is after 12 min. Therefore, unbound CJC-1134 has been effectively removed from protein species.

FIG. 11 presents butyl sepharose hydrophobic interaction chromatography of a conjugation reaction between DAC-Exendin-4 (CJC-1134) and recombinant human albumin following a first phenyl sepharose flow through purification. Butyl-Sepharose resin was equilibrated in 20 mM sodium phosphate (pH 7), 5 mM sodium octanoate, and 750 mM ammonium sulfate.

FIG. 12 presents an HPLC chromatogram of unbound DAC-GLP-1 (CJC-1131) found post-conjugation between DAC-GLP-1 (CJC-1131) and rHA prior to loading onto Phenyl-Sepharose flow-through column. Retention time of unbound CJC-1131 is 27.5 min, and that of the albumin conjugate is after 50 min.

FIG. 13 presents phenyl sepharose hydrophobic interaction chromatography of a conjugation reaction between DAC-GLP-1 (CJC-1131) and recombinant human albumin. Phenyl-Sepharose was pre-equilibrated in 20 mM sodium phosphate buffer (pH 7.0) composed of 5 mM sodium octanoote and 5 mM ammonium sulfate. Direct loading of conjugation reaction onto this resin enables physical separation of protein (albumin and conjugated albumin) observed in flow-through from unbound DAC-GLP-1 (CJC-1131). Therefore, capacity of this resin is reserved primarily for unbound compound composed of a reactive moiety.

FIG. 14 presents an HPLC chromatogram of unbound DAC-GLP-1 found post-conjugation between DAC-GLP-1 (CJC-1131) and rHA following loading of reaction mixture onto Phenyl-Sepharose flow-through column. Retention time of unbound CJC-1131 is 27.5 min, and that of the albumin conjugate is after 46 min. Therefore, unbound CJC-1131 has been effectively removed from protein species. [Note: Peak with retention time of 20.5 min corresponds to octanoate.]

FIG. 15 presents a Coomasssie stained gel of recombinant human albumin (lane 3) and a GLP-albumin conjugate (lane 4);

FIG. 16 presents immunodetection of albumin in samples of recombinant human albumin (lane 3) and a GLP-albumin conjugate (lane 4);

FIG. 17 presents Coomassie staining of phenyl and butyl sepharose fractions from purification of a conjugation reaction between DAC-GLP-1 and recombinant human albumin. Lanes are as follows: (1) rHA; (2) Pre-purification; (3) Phenyl F8; (4) Butyl F3 750 mM (NH₄)2SO₄; (5) Butyl F5 550 mM (NH₄)₂SO₄; (6) Butyl F6A 100 mM (NH₄)₂SO₄ before PC 200-2000 mAU; (7) Butyl F6B 100 mM (NH₄)₂SO₄ PC WFI; (8) Butyl F6B 100 mM (NH₄)₂SO₄ PC Acetate; and (9) Standard.

FIG. 18 presents GLP-1 immunodetection of phenyl and butyl sepharose fractions from purification of a conjugation reaction between DAC-GLP-1 and recombinant human albumin.

### 5. DETAILED DESCRIPTION OF THE INVENTION

### 5.1 Definitions

As used herein, “albumin” refers to any serum albumin known to those of skill in the art. Albumin is the most abundant protein in blood plasma having a molecular weight of approximately between 65 and 67 kilodaltons in its monomeric form, depending on the species of origin. The term “albumin” is used interchangeably with “serum albumin” and is not meant to define the source of albumin which forms a conjugate according to the processes of the invention.

As used herein, “therapeutic peptides” are amino acid chains of between 2-50 amino acids with therapeutic activity, as defined below. Each therapeutic peptide has an amino terminus (also referred to as N-terminus or amino terminal amino acid), a carboxyl terminus (also referred to as C-terminus terminal carboxyl terminal amino acid) and internal amino acids located between the amino terminus and the carboxyl terminus. The amino terminus is defined by the only amino acid in the therapeutic peptide chain with a free α-amino group. The carboxyl terminus is defined by the only amino acid in the therapeutic peptide chain with a free α-carboxyl group. In some embodiments, the carboxy terminus may be amidated.

### 5.2 Embodiments of the Invention

The present invention provides processes for the production of preformed albumin conjugates. In particular, the invention provides processes for the in-vitro conjugation of a therapeutic compound to recombinant albumin, wherein a therapeutic compound comprising a reactive group is contacted to recombinant albumin in solution to form a conjugate.

The processes provide for the in-vitro conjugation to albumin in albumin solutions having varying degrees of heterogeneity. In some embodiments, the albumin solution is a liquid medium derived from a host organism. In some embodiments, the albumin solution is a liquid culture. In some embodiments, the albumin solution is a crude lysate. In some embodiments, the albumin solution is a clarified lysate. In some embodiments, the albumin solution is a purified albumin solution. In some embodiments, the albumin solution is a purified albumin solution enriched for mercaptalbumin. In some embodiments, the albumin solution is a purified deglycated albumin solution.

The resulting conjugate is purified by chromatography, for instance hydrophobic interaction chromatography comprising phenyl sepharose and butyl sepharose chromatography, optionally followed by ultrafiltration.

### 5.3 Therapeutic Compounds

### 5.3.1 Therapeutic Groups

Conjugates formed by the processes described herein comprise recombinant albumin covalently bound to a compound comprising a therapeutic group and a reactive moiety. In some embodiments, any therapeutic molecule known to those of skill in the art may comprise the therapeutic group of the compound. In some embodiments, the therapeutic molecule is selected from the group consisting of a peptide, a protein, an organic molecule, RNA, DNA, and a combination thereof. In some embodiments, the therapeutic molecule is a small molecule, such as vinorelbine, gemcitabine, doxorubicin, or paclitaxel.

In particular embodiments of the invention, the therapeutic molecule is a therapeutic peptide or protein. In some embodiments, the therapeutic peptide comprises a peptide having a molecular weight of less than 30 kDa. Exemplary therapeutic peptides include anti-obesity peptides, for example, peptide YY, described in U.S. patent application Ser. No. 11/067,556 (publication no. US 2005/176643), the contents of which are hereby incorporated by reference in its entirety. In some embodiments, the therapeutic peptide is a natriuretic peptide, for example, atrial natriuretic peptide (ANP) or brain natriuretic peptide (BNP), both of which are described in U.S. patent application Ser. No. 10/989,397 (publication no. US 2005/089514), the contents of which are hereby incorporated in its entirety. In some embodiments, the therapeutic peptide is growth hormone releasing factor (GRF), described in U.S. patent application Ser. No. 10/203,809 (publication no. US 2003/073630), the contents of which are hereby incorporated by reference in its entirety. In some embodiments, the therapeutic peptide is an anti-fusiogenic peptide, for example T-20, C34 or T-1249. Other useful peptides include insulin, dynorphin, Kringle 5, TPO, T-118, and urocortin.

In particular embodiments, the therapeutic peptide is an insulinotropic peptide. Insulinotropic peptides include glucagon-like peptide 1 (GLP-1), exendin-3 and exendin-4, and their precursors, derivatives and fragments. Such insulinotropic peptides include those disclosed in U.S. Pat. Nos. 6,514,500; 6,821,949; 6,887,849; 6,849,714; 6,329,336; 6,924,264; and 6,593,295, and international publication no. WO 03/103572, the contents of which are hereby incorporated by reference in their entireties. In some embodiments, the therapeutic peptide is GLP-1. In some embodiments, the therapeutic peptide is a GLP-1 derivative. In some embodiments, the therapeutic peptide is exendin-3. In some embodiments, the therapeutic peptide is an exendin-3 derivative. In some embodiments, the therapeutic peptide is exendin-4. In some embodiments, the therapeutic peptide is an exendin-4 derivative. In some embodiments, the therapeutic peptide is exendin-4(1-39). In some embodiments, the therapeutic peptide is exendin-4(1-39)Lys40. In some embodiments, the therapeutic peptide is GRF. In some embodiments, the therapeutic peptide is a GRF derivative. In some embodiments, the therapeutic peptide is the native GRF peptide sequence (1-29) or (1-44) containing the following mutations, either independently or in combination: D-alanine at position 2; glutamine at position 8; D-arginine at position 11; (N-Me)Lys at position 12; alanine at position 15; and leucine at position 27. In some embodiments, the therapeutic peptide is GRF(D-ala2 gly8 ala15 leu27)Lys30.

In certain embodiments, derivative of a therapeutic peptide includes one or more amino acid substitutions, deletions, and/or additions that are not present in the naturally occurring peptide. Preferably, the number of amino acids substituted, deleted, or added is 1, 2, 3, 4, 5, 6, 7, 8, 9, or 10 amino acids. In one embodiment, such a derivative contains one or more amino acid deletions, substitutions, or additions at the amino and/or carboxy terminal end of the peptide. In another embodiment, such a derivative contains one or more amino acid deletions, substitutions, or additions at any residue within the length of the peptide.

In certain embodiments, the amino acid substitutions may be conservative or non-conservative amino acid substitutions. Conservative amino acid substitutions are made on the basis of similarity in polarity, charge, solubility, hydrophobicity, hydrophilicity, and/or the amphipathic nature of the amino acid residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. In addition, glycine and proline are residues that can influence chain orientation. Non-conservative substitutions will entail exchanging a member of one of these classes for another class.

In certain embodiments, an amino acid substitution may be a substitution with a non-classical amino acid or chemical amino acid analog. Non-classical amino acids include, but are not limited to, the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general.

In certain embodiments, a derivative of a therapeutic peptide shares an overall sequence homology with the peptide of at least 75%, at least 85%, or at least 95%. Percent homology in this context means the percentage of amino acid residues in the candidate sequence that are identical (i.e., the amino acid residues at a given position in the alignment are the same residue) or similar (i.e., the amino acid substitution at a given position in the alignment is a conservative substitution, as discussed above), to the corresponding amino acid residue in the peptide after aligning the sequences and introducing gaps, if necessary, to achieve the maximum percent sequence homology In certain embodiments, a derivative of a therapeutic peptide is characterized by its percent sequence identity or percent sequence similarity with the peptide. Sequence homology, including percentages of sequence identity and similarity, are determined using sequence alignment techniques well-known in the art, preferably computer algorithms designed for this purpose, using the default parameters of said computer algorithms or the software packages containing them.

Nonlimiting examples of computer algorithms and software packages incorporating such algorithms include the following. The BLAST family of programs exemplify a preferred, non-limiting example of a mathematical algorithm utilized for the comparison of two sequences (e.g., Karlin & Altschul, 1990, Proc. Natl. Acad. Sci. USA 87:2264-2268 (modified as in Karlin & Altschul, 1993, Proc. Natl. Acad. Sci. USA 90:5873-5877), Altschul et al., 1990, J. Mol. Biol. 215:403-410, (describing NBLAST and XBLAST), Altschul et al., 1997, Nucleic Acids Res. 25:3389-3402 (describing Gapped BLAST, and PSI-Blast). Another preferred example is the algorithm of Myers and Miller (1988 CABIOS 4:11-17) which is incorporated into the ALIGN program (version 2.0) and is available as part of the GCG sequence alignment software package. Also preferred is the FASTA program (Pearson W. R. and Lipman D. J., Proc. Nat. Acad. Sci. USA, 85:2444-2448, 1988), available as part of the Wisconsin Sequence Analysis Package. Additional examples include BESTFIT, which uses the “local homology” algorithm of Smith and Waterman (Advances in Applied Mathematics, 2:482-489, 1981) to find best single region of similarity between two sequences, and which is preferable where the two sequences being compared are dissimilar in length; and GAP, which aligns two sequences by finding a “maximum similarity” according to the algorithm of Neddleman and Wunsch (J. Mol. Biol. 48:443-354, 1970), and is preferable where the two sequences are approximately the same length and an alignment is expected over the entire length.

In certain embodiments, a derivative of a therapeutic peptide shares a primary amino acid sequence homology over the entire length of the sequence, without gaps, of at least 55%, at least 65%, at least 75%, or at least 85% with the peptide. In a preferred embodiment, a derivative of a therapeutic peptide shares a primary amino acid sequence homology over the entire length of the sequence, without gaps, of at least 90% or at least 95% with the peptide.

In a preferred embodiment, the percent identity or similarity is determined by determining the number of identical (for percent identity) or conserved (for percent similarity) amino acids over a region of amino acids, which region is equal to the total length of the shortest of the two peptides being compared (or the total length of both, if the sequence of both are identical in size). In another embodiment, percent identity or similarity is determined using a BLAST algorithm, with default parameters.

### 5.3.1.1 GLP-1 and GLP-1 Derivatives

The hormone glucagon can be synthesized according to any method known to those of skill in the art. In some embodiments, it is synthesized as a high molecular weight precursor molecule which is subsequently proteolytically cleaved into three peptides: glucagon, GLP-1, and glucagon-like peptide 2 (GLP-2). GLP-1 has 37 amino acids in its unprocessed form as shown in SEQ ID NO: 1 (HDEFERHAEG TFTSDVSSYL EGQAAKEFIA WLVKGRG). Unprocessed GLP-1 is essentially unable to mediate the induction of insulin biosynthesis. The unprocessed GLP-1 peptide is, however, naturally converted to a 31-amino acid long peptide (7-37 peptide) having amino acids 7-37 of GLP-1 (“GLP-1(7-37)”) SEQ ID NO:2 (HAEG TFTSDVSSYL EGQAAKEFIA WLVKGRG). GLP-1(7-37) can also undergo additional processing by proteolytic removal of the C-terminal glycine to produce GLP-1(7-36), which also exists predominantly with the C-terminal residue, arginine, in amidated form as arginineamide, GLP-1(7-36) amide. This processing occurs in the intestine and to a much lesser extent in the pancreas, and results in a polypeptide with the insulinotropic activity of GLP-1(7-37).

A compound is said to have an “insulinotropic activity” if it is able to stimulate, or cause the stimulation of, the synthesis or expression of the hormone insulin. The hormonal activity of GLP-1(7-37) and GLP-1(7-36) appear to be specific for the pancreatic beta cells where it appears to induce the biosynthesis of insulin. Glucagon-like-peptide hormones are useful in the study of the pathogenesis of maturity onset diabetes mellitus, a condition characterized by hyperglycemia in which the dynamics of insulin secretion are abnormal. Moreover, glucagon-like peptides are useful in the therapy and treatment of this disease, and in the therapy and treatment of hyperglycemia.

Peptide moieties (fragments) can be chosen from the determined amino acid sequence of human GLP-1. The interchangeable terms “peptide fragment” and “peptide moiety” are meant to include both synthetic and naturally occurring amino acid sequences derivable from a naturally occurring amino acid sequence, or generated using recombinant means.

The amino acid sequence for GLP-1 has been reported by several researchers. See Lopez, L. C. et al., Proc. Natl. Acad. Sci. USA 80:5485-89 (1983); Bell, G. I. et al., Nature 302:716-718 (1983); Heinrich, G. et al., Endocrinol. 115:2176-81 (1984), the contents of which are incorporated by reference. The structure of the preproglucagon mRNA and its corresponding amino acid sequence is well known. The proteolytic processing of the precursor gene product, proglucagon, into glucagon and the two insulinotropic peptides has been characterized. As used herein, the notation of GLP-1(1-37) refers to a GLP-1 polypeptide having all amino acids from 1 (N-terminus) through 37 (C-terminus). Similarly, GLP-1(7-37) refers to a GLP-1 polypeptide having all amino acids from 7 (N-terminus) through 37 (C-terminus). Similarly, GLP-1(7-36) refers to a GLP-1 polypeptide having all amino acids from number 7 (N-terminus) through number 36 (C-terminus).

In one embodiment, GLP-1(7-36) and its peptide fragments are synthesized by conventional means as detailed below, such as by the well-known solid-phase peptide synthesis described by Merrifield, Chem. Soc. 85:21491962 (1962), and Stewart and Young, Solid Phase Peptide Synthesis, Freeman, San Francisco, 1969, pp. 27-66, the contents of which are hereby incorporated by reference. However, it is also possible to obtain fragments of the proglucagon polypeptide, or of GLP-1, by fragmenting the naturally occurring amino acid sequence, using, for example, a proteolytic enzyme. Further, it is possible to obtain the desired fragments of the proglucagon peptide or of GLP-1 through the use of recombinant DNA technology, as disclosed by Maniatis, T., et al., Molecular Biology: A Laboratory Manual, Cold Spring Harbor, N.Y. (1982), the contents of which are hereby incorporated by reference.

Useful peptides for the methods described herein include those which are derivable from GLP-1 such as GLP-1(1-37) and GLP-1(7-36). A peptide is said to be “derivable from a naturally occurring amino acid sequence” if it can be obtained by fragmenting a naturally occurring sequence, or if it can be synthesized based upon a knowledge of the sequence of the naturally occurring amino acid sequence or of the genetic material (DNA or RNA) which encodes this sequence.

Also useful are those molecules which are said to be “derivatives” of GLP-1, such as GLP-1(1-37) and especially GLP-1(7-36). Such a “derivative” has the following characteristics: (1) it shares substantial homology with GLP-1 or a similarly sized fragment of GLP-1; (2) it is capable of functioning as an insulinotropic hormone; and (3) the derivative has an insulinotropic activity of at least 0.1%, 1%, 5%, 10%, 15%, 25% 50%, 75%, 100%, or greater than 100% of the insulinotropic activity of GLP-1.

A derivative of GLP-1 is said to share “substantial homology” with GLP-1 if the amino acid sequences of the derivative is at least 75%, at least 80%, and more preferably at least 90%, and most preferably at least 95%, the same as that of GLP-1(1-37).

Useful derivatives also include GLP-1 derivatives which, in addition to containing a sequence that is substantially homologous to that of a naturally occurring GLP-1 peptide may contain one or more additional amino acids at their amino and/or their carboxy termini, or internally within said sequence. Thus, useful derivatives include polypeptide fragments of GLP-1 that may contain one or more amino acids that may not be present in a naturally occurring GLP-1 sequence provided that such polypeptides have an insulinotropic activity of at least 0.1%, 1%, 5%, 10%, 25% 50%, 75%, 100%, or greater than 100% of the insulinotropic activity of GLP-1. The additional amino acids may be D-amino acids or L-amino acids or combinations thereof.

Useful GLP-1 fragments also include those which, although containing a sequence that is substantially homologous to that of a naturally occurring GLP-1 peptide, lack one or more amino acids at their amino and/or their carboxy termini that are naturally found on a GLP-1 peptide. Thus, useful polypeptide fragments of GLP-1 may lack one or more amino acids that are normally present in a naturally occurring GLP-1 sequence provided that such polypeptides have an insulinotropic activity of at least 0.1%, 1%, 5%, 10%, 25% 50%, 75%, 100%, or greater than 100% of the insulinotropic activity of GLP-1. In certain embodiments, the polypeptide fragments lack one amino acid normally present in a naturally occurring GLP-1 sequence. In some embodiments, the polypeptide fragments lack two amino acids normally present in a naturally occurring GLP-1 sequence. In some embodiments, the polypeptide fragments lack three amino acids normally present in a naturally occurring GLP-1 sequence. In some embodiments, the polypeptide fragments lack four amino acids normally present in a naturally occurring GLP-1 sequence.

Also useful are obvious or trivial variants of the above-described fragments which have inconsequential amino acid substitutions (and thus have amino acid sequences which differ from that of the natural sequence) provided that such variants have an insulinotropic activity which is substantially identical to that of the above-described GLP-1 derivatives.

In addition to those GLP-1 derivatives with insulinotropic activity, GLP-1 derivatives which stimulate glucose uptake by cells but do not stimulate insulin expression or secretion are useful for the methods described herein. Such GLP-1 derivatives are described in U.S. Pat. No. 5,574,008, which is hereby incorporated by reference in its entirety.

GLP-1 derivatives which stimulate glucose uptake by cells but do not stimulate insulin expression or secretion which find use in the methods described herein include:

(SEQ ID NO: 3)
R¹-Ser-Tyr-Leu-Glu-Gly-Gln-Ala-Ala-Lys-Glu-Phe-
Ile-Ala-Trp-Leu-Val-Xaa-Gly-Arg-R²

wherein R¹ is selected from:
a) H₂N; b) H₂N-Ser; c) H₂N-Val-Ser; d) H₂N-Asp-Val-Ser; e) H₂N-Ser-Asp-Val-Ser (SEQ ID NO:4); f) H₂N-Thr-Ser-Asp-Val-Ser (SEQ ID NO:5); g) H₂N-Phe-Thr-Ser-Asp-Val-Ser (SEQ ID NO:6); h) H₂N-Thr-Phe-Thr-Ser-Asp-Val-Ser (SEQ ID NO:7); i) H₂N-Gly-Thr-Phe-Thr-Ser-Asp-Val-Ser (SEQ ID NO:8); j) H₂N-Glu-Gly-Thr-Phe-Thr-Ser-Asp-Val-Ser (SEQ ID NO:9); and, k) H₂N-Ala-Glu-Gly-Thr-Phe-Thr-Ser-Asp-Val-Ser (SEQ ID NO:10); 1) H₂N-His-Ala-Glu-Gly-Thr-Phe-Thr-Ser-Asp-Val-Ser (SEQ ID NO:11); m) H₂N-His-D-Ala-Glu-Gly-Thr-Phe-Thr-Ser-Asp-Val-Ser (SEQ ID NO:12). In the peptide, Xaa is selected from Lys and Arg and R² is selected from NH₂, OH, Gly-NH₂, and Gly-OH.
   These peptides are C-terminal GLP-1 fragments which do not have insulinotropic activity but which are nonetheless useful for treating diabetes and hyperglycemic conditions as described in U.S. Pat. No. 5,574,008, which is hereby incorporated by reference in its entirety.

### 5.3.1.2 Exendin-3 and Exendin-4 Peptides and Their Derivatives

The exendin-3 and exendin-4 peptide can be any exendin-3 or exendin-4 peptide known to those of skill in the art. Exendin-3 and exendin-4 are 39 amino acid peptides (differing at residues 2 and 3) which are approximately 53% homologous to GLP-1 and find use as insulinotropic agents.

The native exendin-3 sequence is HSDGTFTSDLSKQMEEEAVRLFIEWLKNGG PSSGAPPPS (SEQ ID NO:13) and the exendin-4 sequence is HGEGTFTSDLSKQMEEEAVRLFIEWLKNGGPSSGAPPPS (SEQ ID NO:14).

Also useful for the methods described herein are insulinotropic fragments of exendin-4 comprising the amino acid sequences: exendin-4(1-31) (SEQ ID NO:15) HGEGTFTSDLSKQMEEAVRLFIEWLKNGGPY and exendin-4(1-31) (SEQ ID NO:16) HGEGTFTSDLSKQMEEEAVRLFIEWLKNGGY.

Also useful is the inhibitory fragment of native exendin-4 comprising the amino acid sequence: exendin-4(9-39) (SEQ ID NO:17) DLSKQMEEEAVRLFIEWLKNGGPSSGAPPPS.

Other exemplary insulinotropic peptides are presented in SEQ ID NOS:18-24.

SEQ ID NO: 18
HDEFERHAEGTFTSDVSSYLEGQAAKEFIAWLVKGRK
SEQ ID NO: 19
HAEGTFTSDVSSYLEGQAAKEFIAWLVKGRK
SEQ ID NO: 20
HGEGTFTSDLSKQMEEEAVRLFIEWLKNGGPSSGAPPPSK
SEQ ID NO: 21
HSDGTFTSDLSKQMEEEAVRLFIEWLKNGGPSSGAPPPSK
SEQ ID NO: 22
HGEGTFTSDLSKEMEEEVRLFIEWLKNGGPY
SEQ ID NO: 23
HGEGTFTSDLSKEMEEEVRLFIEWLKNGGY
SEQ ID NO: 24
DLSKQMEEEAVRLFIEWLKGGPSSGPPPS

Useful peptides for the processes described herein also include peptides which are derivable from the naturally occurring exendin-3 and exendin-4 peptides. A peptide is said to be “derivable from a naturally occurring amino acid sequence” if it can be obtained by fragmenting a naturally occurring sequence, or if it can be synthesized based upon a knowledge of the sequence of the naturally occurring amino acid sequence or of the genetic material (DNA or RNA) which encodes this sequence.

Useful molecules for the processes described herein also include those which are said to be “derivatives” of exendin-3 and exendin-4. Such a “derivative” has the following characteristics: (1) it shares substantial homology with exendin-3 or exendin-4 or a similarly sized fragment of exendin-3 or exendin-4; (2) it is capable of functioning as an insulinotropic hormone and (3) the derivative has an insulinotropic activity of at least 0.1%, 1%, 5%, 10%, 25% 50%, 75%, 100%, or greater than 100% of the insulinotropic activity of either exendin-3 or exendin-4.

A derivative of exendin-3 and exendin-4 is said to share “substantial homology” with exendin-3 and exendin-4 if the amino acid sequences of the derivative is at least 75%, at least 80%, and more preferably at least 90%, and most preferably at least 95%, the same as that of either exendin-3 or 4 or a fragment of exendin-3 or 4 having the same number of amino acid residues as the derivative.

Useful derivatives also include exendin-3 or exendin-4 fragments which, in addition to containing a sequence that is substantially homologous to that of a naturally occurring exendin-3 or exendin-4 peptide may contain one or more additional amino acids at their amino and/or their carboxy termini, or internally within said sequence. Thus, useful derivatives include polypeptide fragments of exendin-3 or exendin-4 that may contain one or more amino acids that may not be present in a naturally occurring exendin-3 or exendin-4 sequences provided that such polypeptides have an insulinotropic activity of at least 0.1%, 1%, 5%, 10%, 25% 50%, 75%, 100%, or greater than 100% of the insulinotropic activity of either exendin-3 or exendin-4.

Similarly, useful derivatives include exendin-3 or exendin-4 fragments which, although containing a sequence that is substantially homologous to that of a naturally occurring exendin-3 or exendin-4 peptide may lack one or more additional amino acids at their amino and/or their carboxy termini that are naturally found on a exendin-3 or exendin-4 peptide. Thus, useful derivatives include polypeptide fragments of exendin-3 or exendin-4 that may lack one or more amino acids that are normally present in a naturally occurring exendin-3 or exendin-4 sequence, provided that such polypeptides have an insulinotropic activity of at least 0.1%, 1%, 5%, 10%, 25% 50%, 75%, 100%, or greater than 100% of the insulinotropic activity of either exendin-3 or exendin-4.

Useful derivatives also include the obvious or trivial variants of the above-described fragments which have inconsequential amino acid substitutions (and thus have amino acid sequences which differ from that of the natural sequence) provided that such variants have an insulinotropic activity which is substantially identical to that of the above-described exendin-3 or exendin-4 derivatives.

### 5.3.1.3 GRF and GRF Derivatives

Growth hormone (GH), also known as somatotropin, is a protein hormone of about 190 amino acids synthesized and secreted by cells called somatotrophs in the anterior pituitary. It is a major participant in control of growth and metabolism. It is also of considerable interest as a pharmaceutical product for use in both humans and animals. The production of GH is modulated by many factors, including stress, nutrition, sleep and GH itself. However, its primary controllers are two hypothalamic hormones: the growth hormone-releasing factor (GRF or GHRH), a 44 amino acid sequence that stimulates the synthesis and secretion of GH and; somatostatin (SS), which inhibits GH release in response to GRF.

It has been shown that the biological activity of GRF (1-44) resides in the N terminal portion of the peptide. Full intrinsic activity and potency was also demonstrated with GRF (1-29) both in vitro and in vivo. Furthermore, sustained administration of GRF induces the same episodic secretory pattern of GH from the pituitary gland as under normal physiological conditions. Thus GRF has great therapeutic utility in those instances where growth hormone is indicated. For example, it may be used in the treatment of hypopituitary dwarfism, diabetes due to GH production abnormalities, and retardation of the aging process. Many other diseases or conditions benefiting from endogenous production or release of GRF are enumerated below. Further, GRF is useful in the field of agriculture. Examples of agricultural uses include enhanced meat production of pigs, cattle or the like to permit earlier marketing. GRF is also known to stimulate milk production in dairy cows. Other exemplary applications are described in U.S. patent application Ser. No. 10/203,809 (publication no. US 2003/073630), the contents of which are hereby incorporated by reference in its entirety.

Thus, in certain embodiments, conjugates comprising GRF as a therapeutic peptide may be formed by the processes of the invention. Useful peptides also include GRF derivatives which, although containing a sequence that is substantially homologous to that of a naturally occurring GRF peptide, may lack one or more additional amino acids at their amino and/or their carboxy termini that are naturally found on a GRF native peptide. Thus, useful peptides include polypeptide fragments of GRF that may lack one or more amino acids that are normally present in a naturally occurring GRF sequence, provided that such polypeptides have growth hormone releasing activity of at least 0.1%, 1%, 5%, 10%, 25%, 50%, 75%, 100% or greater than 100% of the growth hormone releasing activity of GRF.

A derivative of GRF is said to share “substantial homology” with GRF if the amino acid sequences of the derivative is at least 75%, at least 80%, and more preferably at least 90%, and most preferably at least 95%, the same as that of GRF.

Useful peptides for the processes described herein also include the obvious or trivial variants of the above-described analogs or fragments which have inconsequential amino acid substitutions (and thus have amino acid sequences which differ from that of the natural sequence) provided that such variants have growth hormone releasing activity which is at least 0.1%, 1%, 5%, 10%, 25%, 50%, 75%, 100% or greater than 100% of the growth hormone releasing activity of GRF.

In a particular embodiment, the GRF peptide sequence useful for the processes described herein is of the following sequence:
A₁-A₂-Asp-A₄-Ile-Phe-A₇-A₈-A₉-Tyr-A₁₁-A₁₂-A₁₃-Leu-A₁₅-Gln-Leu-A₁₈-Ala-A₂₀-A₂₁-A₂₂-LeU-A₂₄-A₂₅-A₂₆-A₂₇-A₂₈-A₂₉-A₃₀
wherein,
A₁ is Tyr, N-Ac-Tyr, His, 3-MeHis, desNH₂ His, desNH₂ Tyr, Lys-Tyr, Lys-His or Lys-3-MeHis;
A₂ is Val, Leu, Ile, Ala, D-Ala, N-methyl-D-Ala, (N-methyl)-Ala, Gly, Nle ou Nval;
A₄ is Ala or Gly;
A₅ is Met or Ile;
A₇ is Asn, Ser or Thr;
A₈ is Asn, Gln, Lys or Ser;
A₉ is Ala or Ser;
A₁₁ is Arg, D-Arg, Lys or D-Lys;
A₁₂ is Lys, (N-Me)Lys, or D-Lys;
A₁₃ is Val or Leu;
A₁₅ is Ala, Leu or Gly;
A₁₈ is Ser or Thr;
A₂₀ is Arg, D-Arg, Lys or D-Lys;
A₂₁ is Lys, (N-Me)Lys, or Asn;
A₂₂ is Tyr or Leu;
A₂₄ is Gln or His;
A₂₅ is Ser or Asp;
A₂₆ is Leu or Ile;
A₂₇ is Met, Ile, Leu or Nie;
A₂₈ is Ser, Asn, Ala or Asp;
A₂₉ is Lys or Arg; and
A₃₀ is absent, X, or X-Lys wherein X is absent or is the sequence Gln-Gln-Gly-Glu-Ser-Asn-Gln-Glu-Arg-Gly-Ala-Arg-Ala-Arg-Leu or a fragment thereof,
wherein the fragment is reduced by one to fifteen amino acids from the C-terminal; and wherein one amino acid residue from the fragment can optionally be replaced with a lysine residue; and wherein the C-terminal can be the free carboxylic acid or the corresponding amide,
   with the proviso that if A₂ is Ala, then the fragment is not a fragment reduced by 5-8 amino acids.

In addition to promoting endogenous production or release of growth hormone, the present GRF derivatives may incorporate an amino acid substitution at one or more sites within a GRF peptide “backbone”, or is a variant of GRF species in which the C-terminal and/or the N-terminal has been altered by addition of one or more basic residues, or has been modified to incorporate a blocking group of the type used conventionally in the art of peptide chemistry to protect peptide termini from undesired biochemical attack and degradation in vivo. Thus, the present GRF derivatives incorporate an amino acid substitution in the context of any GRF species, including but not limited to human GRF, bovine GRF, rat GRF, porcine GRF etc., the sequences of which having been reported by many authors. In a more preferred embodiment, a lysine residue is added at the C-terminal or N-terminal of the GRF peptide sequence.

### 5.4 Reactive Groups

In preferred embodiments, conjugates formed by the processes described herein comprise a therapeutic molecule covalently joined to recombinant albumin via a reactive group. The reactive group is chosen for its ability to form a stable covalent bond with albumin, for example, by reacting with one or more amino groups, hydroxyl groups, or thiol groups on albumin. Preferably, a reactive group reacts with only one amino group, hydroxyl group, or thiol group on albumin. Preferably, a reactive group reacts with a specific amino group, hydroxyl group, or thiol group on albumin. In some embodiments, conjugates formed by the processes described herein comprise a therapeutic peptide, or a modified derivative thereof, which is covalently attached to albumin via a reaction of the reactive group with an amino group, hydroxyl group, or thiol group on albumin. Thus, a conjugate formed by the processes of the invention may comprise a therapeutic peptide, or a modified derivative thereof, in which the reactive group has formed a covalent bond to albumin. Even more preferably, the reactive group forms a covalent bond with the Cys34 thiol of albumin.

To form covalent bonds with the functional group on a protein, one may use as a chemically reactive group a wide variety of active carboxyl groups, particularly esters. The carboxyl groups are usually converted into reactive intermediates such as N-hydroxysuccinimide (NHS) or maleimide that are susceptible to attack by amines, thiols and hydroxyl functionalities on the protein. Introduction of NHS and maleimide reactive groups on the peptide can be performed by the use of bifunctionnal linking agents such as maleimide-benzoyl-succinimide (MBS), gamma-maleimido-butyryloxy succinimide ester (GMBS), dithiobis-N-hydrohy succinimido propropionate (DTSP), succinimidyl 3(2-pyridyldithio propionate) (SPDP), succinimidyl trans-4-(maleimidylmethyl)cyclohexane-1-carboxylate (SMCC), succinimidyl acetylthioacetate (SATA), benzophenone 4-maleimide, N-((2-pyridyldithio)ethyl)-4-azidosalicylamide (PEAS; AET). Such bifunctionnal linkers will activate either carboxy or amino groups on the peptide based on the choice of protecting groups.

Alternatively the addition of maleimide to the peptide can be performed through the use of coupling agents such as N,N, dicyclohexylcarbodiimide (DCC), 1-ethyl-3-(3-dimethylaminopropyl)carbodiimide, hydrochloride (EDAC) and the likes to activate derivatives like maleimidopropionic acid, [2-[2-[2-maleimidopropionamido(ethoxy)ethoxy]acetic acid, and subsequently react with an amine on the peptide. Similar agents like DCC and EDAC could also be used to add derivatives like maleimidoalkyl amines to carboxy moieties on the peptide.

Primary amines are the principal targets for NHS esters. Accessible ε-amine groups present on the N-termini of proteins react with NHS esters. However, ε-amino groups on a protein may not be desirable or available for the NHS coupling. While five amino acids have nitrogen in their side chains, only the ε-amine of lysine reacts significantly with NHS esters. An amide bond can form when the NHS ester conjugation reaction reacts with primary amines releasing N-hydroxysuccinimide. These succinimidyl-containing reactive groups are herein referred to as succinimidyl groups.

In particular embodiments, the functional group on albumin is the single free thiol group located at amino acid residue 34 (Cys34) and the chemically reactive group is a maleimido-containing group such as MPA. MPA stands for maleimido propionic acid or maleimidopropionate. Such maleimido-containing groups are referred to herein as maleimido groups.

In some embodiments, conjugates formed by the processes described herein comprise albumin covalently linked to a succinimidyl or maleimido group on a therapeutic peptide. In some embodiments, an albumin amino, hydroxyl or thiol group is covalently linked to a succinimidyl or maleimido group on the therapeutic peptide. In some embodiments, albumin cysteine 34 thiol is covalently linked to a [2-[2-[2-maleimidopropionamido(ethoxy)ethoxy]acetamide linker on the epsilon amino of a lysine of the therapeutic peptide.

In a specific embodiment, the reactive group is a single MPA reactive group attached to the peptide, optionally through a linking group, at a single defined amino acid and the MPA is covalently attached to albumin at a single amino acid residue of albumin, preferably cysteine 34. In a preferred embodiment, the albumin is recombinant human albumin.

In certain embodiments, the reactive group, preferably MPA, is attached to the peptide through one or more linking groups, preferably AEEA, AEA, or octanoic acid. In certain examples of embodiments in which the reactive group, preferably MPA, is attached to the peptide through more than one linking group, each linking group can be independently selected from the group consisting preferably of AEA ((2-amino)ethoxy acetic acid), AEEA ([2-(2-amino)ethoxy)]ethoxy acetic acid), and octanoic acid. In one embodiment, the reactive group, preferably MPA, is attached to the peptide via 0, 1, 2, 3, 4, 5 or 6 AEEA linking groups which are arranged in tandem. In another embodiment, the reactive group, preferably MPA, is attached to the peptide via 0, 1, 2, 3, 4, 5 or 6 octanoic acid linking groups which are arranged in tandem. In certain embodiments, a linking group can comprise, for example, a chain of 0-30 atoms, or 0-20 atoms, or 0-10 atoms. In certain embodiments, a linking group can consist of, for example, a chain of 0-30 atoms, or 0-20 atoms, or 0-10 atoms. Those atoms can be selected from the group consisting of, for example, C, N, O, S, P.

In certain embodiments, the reactive group can be attached to any residue of the therapeutic peptide suitable for attachment of such a reactive group. The residue can be a terminal or internal residue of the peptide. In certain embodiments, the reactive group can be attached to the carboxy-terminus or amino-terminus of the peptide. In advantageous embodiments, the reactive group is attached to a single site of the peptide. This can be achieved using protecting groups known to those of skill in the art. In certain embodiments, a derivative of the therapeutic peptide can comprise a residue incorporated for attachment of the reactive group. Useful residues for attachment include, but are not limited to, lysine, aspartate and glutamate residues. The residue can be incorporated internally or at a terminus of the peptide, for example on the N-terminal amino-acid residue via the free α-amino end. In certain embodiments, the reactive group is attached to an internal lysine residue. In certain embodiments, the reactive group is attached to a terminal lysine residue. In certain embodiments, the reactive group is attached to an amino-terminal lysine residue. In certain embodiments, the reactive group is attached to a carboxy-terminal lysine residue, for instance, a lysine residue at the carboxy-terminus of GLP-1, GLP-1(7-37) or exendin-4.

In other embodiments, an activated disulfide bond group may be coupled to a therapeutic peptide cysteine or cysteine analog through a method for the preferential formation of intermolecular disulfide bonds based on a selective thiol activation scheme. Methods based on the selective activation of one thiol with an activating group followed by a reaction with a second free thiol to form asymmetric disulfide bonds selectively between proteins or peptides have been described to alleviate the problem of reduced yields due to symmetric disulfide bond formation. See D. Andreu et al., “Methods in Molecular Biology” (M. W. Pennington and B. M. Dunn, eds.), Vol. 35, p. 91. Humana Press, Totowa, N.J., (1994), the contents of which are hereby incorporated by reference in its entirety. Preferably, such activating groups are those based on the pyridine-sulfenyl group (M. S. Bernatowicz et al., Int. J. Pept. Protein Res. 28:107(1986)). Preferably, 2,2′-dithiodipyridine (DTDP) (Carlsson et al., Biochem. J. 173: 723(1978); L. H. Kondejewski et al., Bioconjugate Chem. 5:602(1994) or 2,2′-dithiobis(5-Nitropyridine) (NPYS) (J Org. Chem. 56: 6477(1991)) is employed. In addition, 5,5′-dithiobis(2-nitrobenzoic acid) (Ellman's reagent) or 6,6′-dithiodinicotinic acid may be used as activating groups

In accordance with these methods, a disulfide bond activating group is first reacted with a therapeutic peptide containing a cysteine or cysteine analog under conditions of excess activating group. These conditions highly favor the formation of the therapeutic compound containing a therapeutic peptide coupled with an activated disulfide group, with essentially no production of disulfide-bonded peptide homodimers. Following the coupling reaction, the resulting peptide compound is purified, such as by reversed phase-HPLC. A reaction with a second free thiol occurs when the peptide compound is reacted with a blood component, preferably serum albumin, to form a conjugate between the therapeutic compound and serum albumin.

A therapeutic peptide cysteine or cysteine analog is converted to having an S-sulfonate through a sulfitolysis reaction scheme. In this scheme, a therapeutic peptide is first synthesized either synthetically or recombinantly. A sulfitolysis reaction is then used to attach a S-sulfonate to the therapeutic peptide through its cysteine or cysteine analog thiol. Following the sulfitolysis reaction, the therapeutic peptide compound is purified, such as by gradient column chromatography. The S-sulfonate compound is then used to form a conjugate between the therapeutic peptide compound and a blood component, preferably serum albumin.

The manner of modifying therapeutic peptides with a reactive group for conjugation to albumin will vary widely, depending upon the nature of the various elements comprising the therapeutic peptide. The synthetic procedures will be selected so as to be simple, provide for high yields, and allow for a highly purified product. Normally, the chemically reactive group will be created at the last stage of peptide synthesis, for example, with a carboxyl group, esterification to form an active ester. Specific methods for the production of modified insulinotropic peptides are described in U.S. Pat. Nos. 6, 329,336, 6,849,714 or 6,887,849, the contents of which are hereby incorporated by reference in their entirety.

### 5.5 Albumin

Any albumin known to those of skill in the art may be used to form a conjugate according to the processes of the invention. In some embodiments, the albumin may be serum albumin isolated from a host species and purified for use in the formation of a conjugate. The serum albumin may be any mammalian serum albumin known to those of skill in the art, including but not limited to mouse, rat, rabbit, guinea pig, dog, cat, sheep, bovine, ovine, equine, or human albumin. In some embodiments, the albumin is human serum albumin.

While the processes of the invention can be utilized to form albumin conjugates comprising albumin from a number of sources, such as serum or a genomic source, the processes are particularly applicable to forming conjugates with recombinant albumin. The recombinant albumin may be any mammalian albumin known to those of skill in the art, including but not limited to mouse, rat, rabbit, guinea pig, dog, cat, sheep, bovine, ovine, equine, or human albumin. In a preferred embodiment, the recombinant albumin is recombinant human albumin, in particular, recombinant human serum albumin (rHSA).

Human serum albumin (HSA) is responsible for a significant proportion of the osmotic pressure of serum and also functions as a carrier of endogenous and exogenous ligands. In its mature form, HSA is a non-glycosylated monomeric protein of 585 amino acids, corresponding to a molecular weight of about 66 kD. Its globular structure is maintained by 17 disulfide bridges which create a sequential series of 9 double loops. See Brown, J. R., Albumin Structure, Function and Uses, Rosenoer, V. M. et al. (eds), Pergamon Press, Oxford (1977), the contents of which are hereby incorporated by reference in its entirety. Thus, conjugates formed with the mature form of albumin are within the scope of the processes described herein.

In some embodiments, conjugates formed by the processes of the invention comprise an albumin precursor. Human albumin is synthesized in liver hepatocytes and then secreted in the blood stream. This synthesis leads, in a first instance, to a precursor, prepro-HSA, which comprises a signal sequence of 18 amino acids directing the nascent polypeptide into the secretory pathway. Thus, conjugates formed with an albumin precursor are within the scope of the processes described herein.

In certain embodiments, conjugates formed by the processes of the invention comprise molecular variants of albumin. Variants of albumin may include natural variants resulting from the polymorphism of albumin in the human population. More than 30 apparently different genetic variants of human serum albumin have been identified by electrophoretic analysis under various conditions. See e.g., Weitkamp et al., Ann. Hum. Genet., 36(4):381-92 (1973); Weitkamp, Isr. J. Med. Sci., 9(9):1238-48 (1973); Fine et al., Biomedicine, 25(8):291-4 (1976); Fine et al., Rev. Fr. Transfus. Immunohematol., 25(2):149-63. (1982); Rochu et al., Rev. Fr. Transfus. Immunohematol. 31(5):725-33 (1988); Arai et al., Proc. Natl. Acad. Sci. U.S.A 86(2): 434-8 (1989), the contents of which are hereby incorporated by reference in their entirety. Thus, conjugates formed with molecular variants of albumin are within the scope of the processes described herein.

In some embodiments, conjugates formed by the processes of the invention comprise derivatives of albumin which share substantial homology with albumin. For instance, conjugates may be formed with an albumin homologue having an amino acid sequence at least 75%, at least 80%, at least 85%, more preferably at least 90%, and most preferably at least 95%, the same as that of albumin. In certain embodiments, the albumin homologue comprises a free cysteine. In certain embodiments, the albumin homologue comprises a single free cysteine. In some embodiments, the albumin homologue comprises a free cysteine 34.

In some embodiments, conjugates formed by the processes of the invention comprise structural derivatives of albumin. Structural derivatives of albumin may include proteins or peptides which possess an albumin-type activity, for example, a functional fragment of albumin. In some embodiments, the derivative is an antigenic determinant of albumin, i.e., a portion of a polypeptide that can be recognized by an anti-albumin antibody. In some embodiments, the recombinant albumin may be any protein with a high plasma half-life which may be obtained by modification of a gene encoding human serum albumin. By way of example and not limitation, the recombinant albumin may contain insertions or deletions in the trace metal binding region of albumin, such that binding of trace metals, e.g., nickel and/or copper is reduced or eliminated, as described in U.S. Pat. No. 6,787,636, the contents of which are incorporated by reference in their entirety. Reduced trace metal binding by albumin may be advantageous for reducing the likelihood of an allergic reaction to the trace metal in the subject being treated with the albumin composition.

Structural derivatives of albumin may be generated using any method known to those of skill in the art, including but not limited to, oligonucleotide-mediated (site-directed) mutagenesis, alanine scanning, and polymerase chain reaction (PCR) mutagenesis. Site-directed mutagenesis (see Carter, Biochem. J. 237:1-7 (1986); Zoller and Smith, Methods Enzymol. 154:329-50 (1987)), cassette mutagenesis, restriction selection mutagenesis (Wells et al., Gene 34:315-323 (1985)) or other known techniques can be performed on cloned albumin-encoding DNA to produce albumin variant DNA or sequences which encode structural derivatives of albumin (Ausubel et al., Current Protocols In Molecular Biology, John Wiley and Sons, New York (current edition); Sambrook et al., Molecular Cloning, A Laboratory Manual, 3d. ed., Cold Spring Harbor Laboratory Press, Cold Spring Harbor, N.Y. (2001), the contents of which are hereby incorporated by reference in their entirety.

In certain embodiments, albumin derivatives include any macromolecule with a high plasma half-life obtained by in vitro modification of the albumin protein. In some embodiments, the albumin is modified with fatty acids. In some embodiments, the albumin is modified with metal ions. In some embodiments, the albumin is modified with small molecules having high affinity to albumin. In some embodiments, the albumin is modified with sugars, including but not limited to, glucose, lactose, mannose, and galactose.

In some embodiments, conjugates formed by the processes described herein may comprise an albumin fusion protein, i.e., an albumin molecule, or a fragment or variant thereof, fused to a therapeutic protein, or a fragment or variant thereof. The albumin fusion protein may be generated by translation of a nucleic acid comprising a polynucleotide encoding all or a portion of a therapeutic protein joined to a polynucleotide encoding all or a portion of albumin. Any albumin fusion protein known to those of skill in the art may be used to form conjugates according to the processes of the invention. Exemplary albumin fusion proteins are described in U.S. Pat. Nos. 6,548,653, 6,686,179, 6,905,688, 6,994,857, 7,045,318, 7,056,701, and 7,141,547, the contents of which are incorporated herein by reference in their entirety. In some embodiments, the albumin fusion protein is comprised of albumin, or a fragment or variant thereof, fused to a glucagon-like peptide 1 as described in U.S. Pat. No. 7,141,547. In some embodiments, the albumin fusion protein is comprised of albumin, or a fragment or variant thereof, fused to exendin-3, or a fragment or variant thereof. In some embodiments, the albumin fusion protein is comprised of albumin, or a fragment or variant thereof, fused to exendin-4, or a fragment or variant thereof.

Albumin used to form a conjugate according to the present invention may be obtained using methods or materials known to those of skill in the art. For instance, albumin can be obtained from a commercial source, e.g., Novozymes Inc. (Davis, Calif.; recombinant human albumin derived from Saccharomyces cerevisiae); Cortex-Biochem (San Leandro, Calif.; serum albumin), Talecris Biotherapeutics (Research Triangle Park, N.C.; serum albumin), ZLB Behring (King of Prussia, Pa.), or New Century Pharmaceuticals (Huntsville, Ala.; recombinant human albumin derived from Pichia pastoris).

### 5.6 Producing Recombinant Albumin in a Host Cell

In certain embodiments, DNA encoding albumin, or a variant or derivative thereof, may be expressed in a suitable host cell to produce recombinant albumin for conjugation. Thus, expression vectors encoding albumin may be constructed in accordance with any technique known to those of skill in the art to construct an expression vector. The vector may then be used to transform an appropriate host cell for the expression and production of albumin to be used to form a conjugate by the processes described herein.

### 5.6.1 Expression Vectors

Generally, expression vectors are recombinant polynucleotide molecules comprising expression control sequences operatively linked to a nucleotide sequence encoding a polypeptide. Expression vectors can be readily adapted for function in prokaryotes or eukaryotes by inclusion of appropriate promoters, replication sequences, selectable markers, etc. to result in stable transcription and translation of mRNA. Techniques for construction of expression vectors and expression of genes in cells comprising the expression vectors are well known in the art. See, e.g., Sambrook et al., 2001, Molecular Cloning—A Laboratory Manual, 3^{rd} edition, Cold Spring Harbor Laboratory, Cold Spring Harbor, N.Y., and Ausubel et al., eds., Current Edition, Current Protocols in Molecular Biology, Greene Publishing Associates and Wiley Interscience, NY.

A variety of host-vector systems may be utilized to express the albumin-encoding sequence. These include, but are not limited to, mammalian cell systems infected with virus (e.g., vaccinia virus, adenovirus, etc.); insect cell systems infected with virus (e.g., baculovirus); microorganisms such as yeast containing yeast vectors; bacteria transformed with bacteriophage, DNA, plasmid DNA, or cosmid DNA; or human cell lines transfected with plasmid DNA. The expression elements of vectors vary in their strengths and specificities. Depending on the host-vector system utilized, any one of a number of suitable transcription and translation elements may be used. In some embodiments, a human albumin cDNA is expressed. In some embodiments, a molecular variant of albumin is expressed. In some embodiments, an albumin precursor is expressed. In some embodiments, a structural derivative of albumin is expressed. In some embodiments, an albumin fusion protein is expressed.

Expression of albumin may be controlled by any promoter/enhancer element known in the art. In a particular embodiment, the promoter is heterologous to (i.e., not a native promoter of) the specific albumin-encoding gene or nucleic acid sequence. Promoters that may be used to control expression of albumin-encoding genes or nucleic acid sequences in mammalian cells include, but are not limited to, the SV40 early promoter region (Bernoist and Chambon, Nature 290:304-310 (1981)), the promoter contained in the 3′ long terminal repeat of Rous sarcoma virus (Yamamoto et al., Cell 22:787-797 (1980)), the herpes thymidine kinase promoter (Wagner et al., Proc. Natl. Acad. Sci. U.S.A. 78:1441-1445 (1981)), and the regulatory sequences of the metallothionein gene (Brinster et al., Nature 296:39-42 (1982));

Promoters that may be useful in prokaryotic expression vectors include, but are not limited to, the β-lactamase promoter (Villa-Kamaroff et al., Proc. Natl. Acad. Sci. U.S.A. 75:3727-3731 (1978)), or the tat promoter (DeBoer et al., Proc. Natl. Acad. Sci. U.S.A. 80:21-25 (1983)). See also “Useful Proteins From Recombinant Bacteria” in Scientific American, 242:74-94 (1980), the contents of which are hereby incorporated by reference in its entirety.

Promoters that may be useful in plant expression vectors include, but are not limited to, the nopaline synthetase promoter region (Herrera-Estrella et al., Nature 303:209-213 (1983)), the cauliflower mosaic virus 35S RNA promoter (Gardner et al., Nucleic Acids Res. 9:2871 (1981)), and the promoter of the photosynthetic enzyme ribulose biphosphate carboxylase (Herrera-Estrella et al., Nature 310:115-120 (1984)).

Promoter elements useful for expression of albumin in yeast or other fungi include the Ga14 promoter, the ADC (alcohol dehydrogenase) promoter, the PGK (phosphoglycerol kinase) promoter, the alkaline phosphatase promoter, or the AOX1 (alcohol oxidase 1) promoter (Ellis et al., Mol. Cell. Biol. 5:1111-1121 (1985)).

In embodiments of the invention where secretion of the recombinant albumin into the culture medium of the host cell is sought, the expression vector may further comprise a “leader” sequence, located upstream of the sequence encoding albumin, or where appropriate, between the region for initiation of transcription and translation and the coding sequence, which directs the nascent polypeptide in the secretory pathways of the selected host. In some embodiments, the leader sequence may be the natural leader sequence of human serum albumin. In other embodiments, the leader sequence is a heterologous sequence. The choice of the leader sequence used is largely guided by the host organism selected. For example, where the host is yeast, it is possible to use, as a heterologous leader sequence, that of the pheromone factor α, invertase, or acid phosphatase. In a particular embodiment, the leader sequence may be that of the Saccharomyces cerevisiae α factor prepro peptide. See Cregg et al., Biotechnology 11:905-910 (1993); Scorer et al., Gene 136:111-119 (1993). In other embodiments, where the host is bacteria, the leader sequence may be that of α-amylase amy_{BamP} or neutral protease npr_{BamP}. Use of these leader sequences for the secretion of recombinant human serum albumin in Bacillus subtilis is described by Saunders et al., J. Bacteriol. 169(7): 2917-25 (1987), the contents of which are hereby incorporated by reference in its entirety. Alternatively, the Sec pathway for transport of the recombinant albumin into the periplasmic space may be utilized. Sec translocase provides a major pathway of protein translocation from the cytosol across the cytoplasmic membrane in bacteria. See e.g., Pugsley A P, Microbiol. Rev., 57(1):50-108 (1993). SecA ATPase interacts dynamically with SecYEG integral membrane components to drive transmembrane movement of newly synthesized preproteins. The premature proteins contain short signal sequences that allow them to be transported outside the cytoplasm, such as pelB, ompA, and phoA, for efficient secretory production of recombinant proteins in E. coli.

### 5.6.2 Host Cells for Producing Recombinant Albumin

Expression vectors containing albumin-encoding sequences may be introduced into a host cell for the production of recombinant albumin. In some embodiments, any cell capable of producing an exogenous recombinant protein may be useful for the processes described herein.

In some embodiments the host organism can be a bacteria strain, for example Escherichia coli and Bacillus subtilis. In some embodiments, the host organism can be a yeast strain, for example Saccharomyces cerevisiae, Pichia pastoris, Kluyveromyces lactis, Arxula adeninivorans, and Hansenula polymorpha. In a particular embodiment, the host organism is Pichia pastoris.

In some embodiments, the recombinant albumin is produced in an insect cell infected with a virus, e.g., baculovirus. In some embodiments, the recombinant albumin is produced in an animal cell. In certain embodiments, the recombinant albumin is produced by a mammalian cell transformed with a vector or infected with a virus encoding albumin, or a variant or derivative thereof. In certain embodiments, the mammalian cell is COS, CHO, or C127 cells. In a particular embodiment, the mammalian cell is the human retinal cell line PER.C6®.

In some embodiments, recombinant albumin is produced in a transgenic non-human animal. The animal may be a mammal, e.g., an ungulate (e.g., a cow, goat, or sheep), pig, mouse or rabbit. In some embodiments, the recombinant albumin secreted into the milk of the animal, as described in U.S. Pat. No. 5,648,243, the contents of which is hereby incorporated by reference in its entirety. In other embodiments, the recombinant albumin is secreted into the blood of the animal, as described in U.S. Pat. No. 6,949,691, the contents of which are hereby incorporated by reference in its entirety. In other embodiments, the recombinant albumin is secreted into the urine of the animal, as described in U.S. patent application Ser. No. 11/401,390, the contents of which are hereby incorporated by reference in its entirety. Methods for generating transgenic animals via embryo manipulation and microinjection, particularly animals such as mice, have become conventional in the art. See e.g., U.S. Pat. Nos. 4,870,009, 4,736,866 and 4,873,191, the contents of which are incorporated by reference in their entirety hereby. Other non-mice transgenic animals expressing recombinant albumin may be made by similar methods.

In some embodiments, the host organism is a plant cell transformed to express recombinant albumin. Methods for expressing human serum albumin in plant cells are well known in the art. See, e.g., Sijmons et al., Biotechnology 8(3):217-21 (1990); Farran et al., Transgenic Res. 11(4):337-46 (2002); Fernandez-San Millan et al., Plant Biotechnol. J. 1(2):71-9 (2003); Baur et al., Plant Biotechnol. J. 3(3):331-40 (2005); and U.S. patent application Ser. No. 11/406,522; the contents of which are hereby incorporated by reference in their entirety.

### 5.6.3 Transformation of the Host Cell

Expression vectors can be introduced into the host cell for expression by any method known to one of skill in the art without limitation. Such methods include, but are not limited to, e.g., direct uptake of the molecule by a cell from solution; or facilitated uptake through lipofection using, e.g., liposomes or immunoliposomes; particle-mediated transfection; etc. See, e.g., U.S. Pat. No. 5,272,065; Goeddel et al., eds, 1990, Methods in Enzymology, vol. 185, Academic Press, Inc., CA; Krieger, 1990, Gene Transfer and Expression—A Laboratory Manual, Stockton Press, NY; Sambrook et al., 1989, Molecular Cloning—A Laboratory Manual, Cold Spring Harbor Laboratory, NY; and Ausubel et al., eds., Current Edition, Current Protocols in Molecular Biology, Greene Publishing Associates and Wiley Interscience, NY.

In a particular embodiment of the invention, recombinant albumin is produced in a yeast cell, in particular Pichia pastoris. Methods for transforming Pichia are well known in the art. See Hinnen et al., Proc. Natl. Acad. Sci. USA 75:1292-3 (1978); Cregg et al., Mol. Cell. Biol. 5:3376-3385 (1985). Exemplary techniques include but are not limited to, spheroplasting, electroporation, PEG 1000 mediated transformation, or lithium chloride mediated transformation.

### 5.6.4 Expression of Recombinant Albumin

Methods for the amplification, induction, and fermentation of host organisms expressing recombinant proteins are well known in the art. See, e.g. Ausubel et al., eds., Current Edition, Current Protocols in Molecular Biology, Greene Publishing Associates and Wiley Interscience, NY. By way of example and not by limitation, general procedures for the expression of recombinant proteins in yeast, for instance Pichia pastoris are as follows: 25 ml of the appropriate culture medium in a 250 ml baffled flask is inoculated using a single recombinant colony. Cells are grown at 28-30° C. in a shaking incubator (250-300 rpm) until culture reaches an OD600=2-6 (approximately 16-18 hours), wherein the cells are in log-phase growth. Cells may then be harvested by centrifugation at 1500-3000×g for 5 minutes at room temperature. Supernatant may be decanted and cell pellet resuspended to an OD600 of 1.0 in an appropriate medium to induce expression (approximately 100-200 ml). The culture may then be placed in a 1 liter baffled flask with 2 layers of sterile gauze or cheesecloth and returned to an incubator for continued growth. An appropriate inducing agent may be added to the culture every 24 hours to maintain induction. Culture samples may be periodically taken (time points (hours): 0, 6, 12, 24 (1 day), 36, 48 (2 days), 60, 72 (3 days), 84, and 96 (4 days) and used to analyze expression levels to determine the optimal time post-induction to harvest. Cells may then be centrifuged at maximum speed in a tabletop microcentrifuge for 2-3 minutes at room temperature. Where the recombinant protein is secreted, supernatant may be transferred to a separate tube. Supernatant and cell pellets may be stored at −80° C. until ready to assay. For intracellular expression, supernatant may be decanted and cell pellets stored at −80° C. until ready to assay. Supernatants and cell pellets may then be assayed for protein expression by, for instance, Coomassie stained SDS-PAGE and western blot or functional assay.

### 5.7 Purification of Recombinant Albumin from the Host Cell

In one aspect of the invention, the process of producing a conjugate optionally comprises purifying the recombinant albumin from the host organism prior to the conjugation reaction. Although the following steps are presented in sequential order, one of skill in the art will recognize that the order of several steps can be interchanged, for instance, the order of the enrichment of mercaptalbumin step and the deglycation of albumin step, without exceeding the scope of the invention. In certain embodiments, where conjugation to secreted recombinant albumin is desired to occur directly in the culture medium, it is understood that the following purification steps may be omitted, and conjugation may be carried out as described in the sections below.

### 5.7.1 Separation of Host Cells from Culture Media

In certain embodiments, the processes of the invention provide, where the host cell is cultured in a liquid medium and the recombinant albumin is secreted therein, for separation of host cells from the medium prior to the conjugation reaction. Any method known in the art to separate host cells from its culture medium may be used. In some embodiments, host cells may be removed from the culture medium by filtration. In a preferred embodiment, the host cells may be separated from the culture medium by centrifugation. Following separation, the resultant supernatant may be used for further purification of the recombinant albumin contained therein. Optionally, where conjugation is desired to occur directly in the culture supernatant, the following steps may be omitted, and conjugation may be carried out as described in the sections below.

### 5.7.2 Lysis of Host Cells

In certain embodiments, the processes of the invention optionally provide, where the host cell is cultured in a liquid medium and the recombinant albumin is predominantly stored intracellularly, for lysis of the host cells prior to the conjugation reaction. Any method of lysing cells known to those of skill in the art may be used. In some embodiments, host cells may be lysed by a mechanical process, e.g., by use of a high speed blender, vortex, homogenizer, French press, Menton Gaulin press, or sonicator.

In particular embodiments where the host organism is yeast, cell lysis may be achieved by any method known to those of skill in the art for lysing yeast cells. In some embodiments, the cells may be lysed by first converting the cells to spheroplasts by contact with a solution containing lyticase or zymolase, then subjecting the spheroplasts to osmotic shock or Dounce homogenization, or a combination thereof Osmotic shock may be achieved by contact with any low osmotic potential solution known to those of skill in the art. In certain embodiments, osmotic shock may be achieved by contacting the spheroplasts with deionized water. In other embodiments, cell lysis of yeast cells may be achieved by mechanical breakage of the cells by vortexing in the presence of glass beads.

In particular embodiments where the host organism is bacteria, cell lysis may be achieved by any method known to those of skill in the art for lysing bacterial cells. In some embodiments, cell lysis may be achieved by contacting cells with a lysozyme solution in the presence of a chelating agent such as EDTA.

In particular embodiments where albumin is expressed in a bacterial cell, additional steps may need to be taken to obtain properly folded recombinant albumin for conjugation. Eukaryotic proteins expressed in large amounts in bacteria, in particular E. Coli, often precipitate into insoluble aggregates called “inclusion bodies.” See Braun et al., Proc. Natl Acad. Sci. USA 99:2654-59 (2002). Inclusion bodies must be isolated, purified and solubilized with denaturing agents, followed by subsequent renaturation of the constituent protein. Protein refolding methodologies utilizing simple dilution, matrix-assisted methods, and the addition of solutes to renaturing buffers are well known in the art. See, e.g., Cabrita et al., Biotechnol. Annu. Rev. 10:31-50 (2004); Mayer et al., Methods Mol. Med. 94:239-254 (2004); Middelberg, Trends Biotechnol. 20:437-443 (2002); Clark, Curr. Opin. Biotechnol. 9:157-163 (1998); and Clark, Curr. Opin. Biotechnol. 12:202-207 (2001), the contents of which are incorporated hereby in their entirety. Accordingly, any method known to one of skill in the art for recovering and renaturing bacterially-expressed eukaryotic proteins may be used to recover and renature recombinant albumin expressed in bacteria.

Following lysis of the host cells, cell debris and particulate matter may be separated from the crude lysate. Any method known in the art to separate cell debris from a crude lysate may be used. In some embodiments, cell debris and particulate matter may be removed by microfiltration. In a preferred embodiment, removal of debris and particulates is achieved by centrifugation. The resultant clarified lysate may be used for further purification of the recombinant albumin contained therein. Optionally, where conjugation is desired to occur directly in the cleared lysate, the following steps may be omitted, and conjugation may be carried out as described in section 5.8 below.

### 5.7.3 Purification of Recombinant Albumin by Chromatography

In certain embodiments, the processes of the invention optionally provide for the purification of the recombinant albumin by chromatography to remove host proteins and antigens, particulate matter, endotoxins, and the like, prior to the conjugation reaction. In certain embodiments, the chromatography can be any chromatographic method known to those of skill in the art to be useful for purification of proteins. By way of example and not by limitation, the chromatography can be ion exchange chromatography, affinity chromatography, gel filtration chromatography, or hydrophobic interaction chromatography.

In some embodiments, the recombinant albumin is purified by ion exchange chromatography. Any ion exchange resin capable of binding albumin according to the judgment of one of skill in the art may be used. In some embodiments, the ion exchanger is a weakly basic anion exchanger such as diethylaminoethyl (DEAE)-cellulose. In certain embodiments, the DEAE-cellulose resin is equilibrated in 10 mM sodium phosphate buffer, pH 7.0. Following loading and binding to the resin, the albumin may be eluted by applying an increasing salt gradient, either linear or stepwise, or a combination thereof. For instance, the albumin may be eluted by contacting the resin with a solution comprising 20 to 200 mM sodium phosphate buffer, pH 7.0. In some embodiments, the albumin is eluted by contacting the resin with a solution comprising 30-150 mM sodium phosphate buffer, pH 7.0. In some embodiments, the albumin is eluted by contacting the resin with 40 to 125 mM sodium phosphate buffer, pH 7.0. In some embodiments, the albumin is eluted by contacting the resin with 50 to 100 mM sodium phosphate buffer, pH 7.0. In some embodiments, the albumin is eluted by contacting the resin with about 60 mM sodium phosphate buffer, pH 7.0. An exemplary purification of recombinant albumin under these conditions is provided in Example 1 below.

In other embodiments, the ion exchanger is a strongly basic anion exchanger such as Q sepharose. In certain embodiments, the Q sepharose resin is equilibrated in 20 mM Tris-HCl buffer, pH 8.0. Following loading and binding to the resin, the albumin may be eluted by applying an increasing salt gradient, either linear or stepwise, or a combination thereof. For instance, the albumin may be eluted by contacting the resin with a solution comprising 0 to 2 M NaCl, pH 8.0. In some embodiments, the albumin is eluted by contacting the resin with a solution comprising 0.1 to 1 M NaCl, pH 8.0. In some embodiments, the albumin is eluted by contacting the resin with 200 to 900 mM NaCl, pH 8.0. In some embodiments, the albumin is eluted by contacting the resin with 300 to 800 mM NaCl, pH 8.0. In some embodiments, the albumin is eluted by contacting the resin with about 500 mM sodium phosphate buffer, pH 8.0. An exemplary purification of recombinant albumin under these conditions is provided in Example 2 below.

In some embodiments, the recombinant albumin is purified by affinity chromatography. Any affinity chromatography ligand capable of binding albumin according to the judgment of one of skill in the art may be used. In some embodiments, the ligand is Cibacron Blue F3G-A, contained for instance in a HiTrap™ Blue HP column (GE Healthcare, Piscataway, N.J.). In certain embodiments, the ligand is equilibrated in 20 mM Tris-HCl buffer, pH 8.0. As Cibacron Blue F3G-A binds albumin by electrostatic and/or hydrophobic interactions with the aromatic anionic ligand, elution may be achieved by applying an increasing salt gradient, either linearly or stepwise, or a combination thereof. Thus, following loading and binding to the ligand, elution of albumin may be achieved, for instance, by contacting the ligand with a solution comprising 0 to 2 M NaCl, pH 8.0. In some embodiments, the albumin is eluted by contacting the resin with 0.2 to 1.5 mM NaCl, pH 8.0. In some embodiments, the albumin is eluted by contacting the resin with 0.5 to 1.0 mM NaCl, pH 8.0. In some embodiments, the albumin is eluted by contacting the resin with about 750 mM sodium phosphate buffer, pH 8.0. An exemplary purification of recombinant albumin under these conditions is provided in Example 3 below.

In some embodiments, the recombinant albumin is purified by hydrophobic interaction chromatography. Any hydrophobic resin capable of binding albumin according to the judgment of one of skill in the art may be used. Exemplary hydrophobic resins include, but are not limited to, octyl sepharose, phenyl sepharose, and butyl sepharose. In a particular embodiment, the hydrophobic resin is phenyl sepharose. In certain embodiments, the phenyl sepharose resin is equilibrated in, for example, a buffer comprising 20 mM sodium phosphate, 5 mM sodium caprylate, and 750 mM (NH₄)₂SO₄, pH 7.0. Following loading and binding to the resin, the albumin may be eluted by applying a decreasing salt gradient, either linear or stepwise, or a combination thereof. For instance, the albumin may be eluted by contact with a solution comprising 0 to 750 mM (NH₄)₂SO₄. In some embodiments, the albumin is eluted by contact with a solution comprising about 300 to 500 mM (NH₄)₂SO₄. In some embodiments, the albumin is eluted by contact with a solution comprising about 350 to 450 mM (NH₄)₂SO₄. In some embodiments, the albumin is eluted by contact with a solution comprising about 375 to 425 mM (NH₄)₂SO₄. In a certain embodiment, the albumin is eluted by contact with a solution comprising about 400 mM (NH₄)₂SO₄. An exemplary purification of recombinant albumin under these conditions is provided in Example 4 below.

In certain embodiments, eluate containing recombinant albumin may be filtered with a low molecular weight filter to concentrate the sample and wash away residual endotoxin and the like. In some embodiments, ultrafiltration may be carried out with an Amicon® 10 kDa

Millipore filter (Millipore Corporation, Bedford, Mass.). In certain embodiments, the recombinant albumin may be washed with sterile water. In other embodiments the recombinant albumin may be washed with 0.9% saline (154 mM NaCl). In other embodiments the recombinant albumin may be washed with sterile buffer.

In certain embodiments, the albumin solution may be concentrated to about 5-250 mg/ml of total protein, corresponding to about 0.5-25% albumin. In some embodiments, the final concentration of the albumin solution comprises about 5 mg/ml, about 10 mg/ml, about 20 mg/ml, about 40 mg/ml, about 80 mg/ml, about 120 mg/ml, about 150 mg/ml, about 175 mg/ml, about 200 mg/ml, about 225 mg/ml, or about 250 mg/ml total protein. In some embodiments, the albumin solution comprises about 0.5%, about 1%, about 2%, about 4%, about 8%, about 12%, about 15%, about 17.5%, about 20%, or about 25% albumin. The albumin sample may then be reformulated in a desired formulation composition.

The resultant recombinant albumin solution may then be used for further purification of the recombinant albumin, for example, enrichment of mercaptalbumin or deglycation, or both. Optionally, where conjugation is desired to occur directly in the partially purified albumin solution, the following steps may be omitted, and conjugation may be carried out as described in section 5.8 below.

### 5.7.4 Enrichment for Mercaptalbumin

Preparations of human serum albumin, whether serum derived or recombinantly produced, may comprise a heterogeneous mixture of nonmercaptalbumin, i.e., “capped' albumin, and mercaptalbumin, i.e., “uncapped” albumin. The human albumin polypeptide contains 35 cysteinyl residues, of which 34 form 17 stabilizing disulfide bridges. While the cysteine residue at position 34 of mercaptalbumin comprises a free SH group, the same residue in nonmercaptalbumin comprises a mixed disulfide with, for example, cysteine or glutathione, or has undergone oxidation by metal ions or other adducts, thus rendering the thiol group less reactive or unavailable. While not intending to be bound by any particular theory of operation, it is believed that enrichment for mercaptalbumin may yield albumin having advantageous properties for conjugation to a therapeutic compound. In particular, specificity of conjugation is enhanced due to the availability of the thiol group of Cys34 to covalently bind the reactive group of the therapeutic compound. Accordingly, in a preferred embodiment of the invention, the purified recombinant albumin is enriched for mercaptalbumin prior to proceeding with the conjugation reaction.

Generally, the enrichment of mercaptalbumin may be carried out using any technique and under any conditions known to those of skill in the art for converting oxidized or “capped” albumin to mercaptalbumin. In some embodiments, the enrichment is achieved by contacting the recombinant albumin with any agent capable of converting oxidized albumin-Cys34 to reduced albumin-Cys34. In certain embodiments, the agent is dithiothreitol (DTT). In a preferred embodiment, the agent is thioglycolic acid (TGA). In some embodiments, the agent is beta-mercaptoethanol (BME). Generally, the agent is contacted with the recombinant albumin under conditions known to those of skill in the art to be suitable to convert capped albumin-Cys34 to mercaptalbumin. Such conditions include, for example, contacting the recombinant albumin with the agent at suitable pH, at a suitable concentration of the agent, at a suitable temperature, and for a suitable time. Generally, the practitioner having skill in the art will take into account the need to preserve the intrachain disulfide bridges of albumin while reducing albumin-Cys34 from an oxidized state.

In certain embodiments, the recombinant albumin is contacted with TGA at a pH suitable for converting capped albumin to mercaptalbumin according to the judgment of one of skill in the art. In certain embodiments, the recombinant albumin is contacted with TGA at a pH of about 5 to 6, or about 5.2 to 5.8, or about 5.3 to 5.7. In particular embodiments, the recombinant albumin is contacted with TGA at about pH 5.6.

In certain embodiments, the recombinant albumin is contacted with TGA at a concentration suitable for converting capped albumin to mercaptalbumin according to the judgment of one of skill in the art. In certain embodiments, recombinant albumin is contacted with TGA at a concentration of about 1 mM, about 5 mM, about 10 mM, about 20 mM, about 40 mM, about 60 mM, about 80 mM, about 100 mM, about 150 mM, about 200 mM, about 250 mM or about 300 mM in a suitable buffer. In certain embodiments, the concentration of TGA is about 1-300 mM, about 5-250 mM, about 10-200 mM, about 20-150 mM, about 40-100 mM, or about 60-80 mM in a suitable buffer. In particular embodiments, the recombinant albumin is contacted with 75 mM TGA in 250 mM Tris acetate buffer.

In certain embodiments, the recombinant albumin is contacted with TGA at a suitable temperature for converting capped albumin to mercaptalbumin according to the judgment of one of skill in the art. In certain embodiments, recombinant albumin is contacted with TGA at about 0-8° C., about 1-7° C., about 2-6° C., or about 3-5° C. In particular embodiments, the recombinant albumin is contacted with TGA at about 4° C. for a time sufficient to convert capped albumin to mercaptalbumin.

In certain embodiments, the recombinant albumin is contacted with TGA for a suitable length of time for converting capped albumin to mercaptalbumin according to the judgment of one of skill in the art. In certain embodiments, recombinant albumin is contacted with TGA for at least 0.1, 1, 5, 10, 15, 20, 25, or 30 hours. In certain embodiments, the recombinant albumin is contacted with TGA for about 5-30 hours, about 10-25 hours, or about 20-25 hours. In certain embodiments, the recombinant albumin is contacted with TGA for about 8, 16, 24 or 32 hours. In particular embodiments, the recombinant albumin is contacted with 75 mM TGA in 250 mM Tris-acetate buffer, pH 5.6 at about 4° C. for about 20 hours.

In other embodiments, enrichment of mercaptalbumin is achieved by contacting the recombinant albumin with DTT. In certain embodiments, the recombinant albumin is contacted with DTT at a pH suitable for converting capped albumin to mercaptalbumin according to the judgment of one of skill in the art. In certain embodiments, the recombinant albumin is contacted with DTT at a pH of about 7 to 8, or about 7.2 to 7.8, or about 7.3 to 7.7. In particular embodiments, the recombinant albumin is contacted with DTT at about pH 7.6.

In certain embodiments, the recombinant albumin is contacted with DTT at a concentration suitable for converting capped albumin to mercaptalbumin according to the judgment of one of skill in the art. In certain embodiments, recombinant albumin is contacted with DTT at a concentration of about 0.1 mM, about 0.25 mM, about 0.5 mM, about 0.75 mM, about 1.0 mM, about 1.5 mM, about 2.0 mM, about 2.5 mM, about 3.0 mM, about 3.5 mM, about 4.0 mM, or about 5.0 mM, in a suitable buffer. In certain embodiments, the concentration of DTT is about 0.1 to 5.0 mM, about 0.25 to 4 mM, about 0.5 to 3.5 mM, about 0.75 to 3.0 mM, about 1.0 to 2.5 mM, or about 1.5 to 2 mM in a suitable buffer. In particular embodiments, the recombinant albumin is contacted with about 2 mM DTT in 1 mM potassium phosphate buffer.

In certain embodiments, the recombinant albumin is contacted with DTT at a suitable temperature for converting capped albumin to mercaptalbumin according to the judgment of one of skill in the art. In certain embodiments, recombinant albumin is contacted with DTT at about 15-40° C., about 20-35° C., about 20-30° C., or about 23-27° C. In particular embodiments, the recombinant albumin is contacted with DTT at about 23-27° C. for a time sufficient to convert capped albumin to mercaptalbumin.

In certain embodiments, the recombinant albumin is contacted with DTT for a suitable length of time for converting capped albumin to mercaptalbumin according to the judgment of one of skill in the art. In certain embodiments, recombinant albumin is contacted with DTT for at least 1, 2, 3, 4, 5, 10, 15, 20, 25, or 30 minutes. In certain embodiments, the recombinant albumin is contacted with DTT for about 1 to 30 minutes, about 2 to 25 minutes, or about 5 to 10 minutes. In certain embodiments, the recombinant albumin is contacted with DTT for about 1, 5, 10 or 30 minutes. In particular embodiments, the recombinant albumin is contacted with 2 mM DTT in 1 mM potassium phosphate buffer at about 23-27° C. for about 5 minutes.

In another embodiment, mercaptalbumin may be enriched from albumin by chromatography. In certain embodiments, the chromatography can be any chromatographic method known in the art to be useful for purifying proteins. Chromatography may be used either as an independent enrichment step, or in combination with, i.e., immediately following contact of the albumin with TGA or DTT, or a combination thereof. In some embodiments, enrichment of mercaptalbumin by chromatographic methods may comprise any of the chromatographic methods described above for the purification of albumin, including but not limited to, ion exchange, affinity, gel filtration, or hydrophobic interaction chromatography.

In preferred embodiments, the mercaptalbumin is further enriched and purified following contact with TGA or DTT, or a combination thereof, by hydrophobic interaction chromatography. Exemplary hydrophobic resins include, but are not limited to, octyl sepharose, phenyl sepharose, or butyl sepharose. In a preferred embodiment, the resin is phenyl sepharose. In certain embodiments, the phenyl sepharose resin is equilibrated in, for example, a buffer comprising 20 mM sodium phosphate, 5 mM sodium caprylate, and 750 mM (NH₄)₂SO₄, pH 7.0. Following loading and binding to the resin, mercaptalbumin may be separated from capped albumin as well as TGA or DTT by applying a decreasing salt gradient, either linear or stepwise, or a combination thereof. For instance, mercaptalbumin may be eluted by contact with a solution comprising 0 to 750 mM (NH₄)₂SO₄. In some embodiments, the albumin is eluted by contact with a solution comprising about 400 to 600 mM (NH₄)₂SO₄. In some embodiments, the albumin is eluted by contact with a solution comprising about 450 to 550 mM (NH₄)₂SO₄. In some embodiments, the albumin is eluted by contact with a solution comprising about 475 to 525 mM (NH₄)₂SO₄. In a certain embodiment, the albumin is eluted by contact with a solution comprising about 500 mM (NH₄)₂SO₄. Under theses conditions, mercaptalbumin may elute prior to capped albumin. An exemplary purification of mercaptalbumin under these conditions is provided in example 5 below.

In certain embodiments, eluate containing recombinant albumin may be filtered with a low molecular weight filter to concentrate the sample and wash away residual endotoxin and the like. In some embodiments, ultrafiltration may be carried out with an Amicon® 10 kDa Millipore filter (Millipore Corporation, Bedford, Mass.). In certain embodiments, the recombinant albumin may be washed with sterile water. In other embodiments the recombinant albumin may be washed with 0.9% saline (154 mM NaCl).

In certain embodiments, the albumin solution may be concentrated to about 5-250 mg/ml of total protein, corresponding to about 0.5-25% albumin. In some embodiments, the final concentration of the albumin solution comprises about 5 mg/ml, about 10 mg/ml, about 20 mg/ml, about 40 mg/ml, about 80 mg/ml, about 120 mg/ml, about 150 mg/ml, about 175 mg/ml, about 200 mg/ml, about 225 mg/ml, or about 250 mg/ml total protein. In some embodiments, the albumin solution comprises about 0.5%, about 1%, about 2%, about 4%, about 8%, about 12%, about 15%, about 17.5%, about 20%, or about 25% albumin. The albumin sample may then be reformulated in a desired formulation composition.

Characterization of the ratio of mercaptalbumin to capped albumin in solution may be carried out by liquid chromatography/mass spectrometry, for example by the methods described by Kleinova et al., Rapid Commun. Mass Spectrom. 19:2965-73 (2005), the contents of which are hereby incorporated by reference in their entirety.

The resultant mercaptalbumin-enriched albumin solution may then be used for further purification, for example reduction of non-enzymatically glycated species of albumin, prior to the conjugation reaction. Optionally, where conjugation is desired to occur directly in the mercaptalbumin solution, the following steps may be omitted, and conjugation may be carried out as described in section 5.8 below.

### 5.7.5 Deglycation of Albumin

In certain embodiments of the invention relating to the production of recombinant albumin in a host organism, in particular yeast strains such as S. cerevisiae and Pichia pastoris, further steps may be taken to limit the level of impurities associated with the recombinant albumin product. In particular, potential differences in the glycosylation profiles of recombinant human albumin compared to serum-derived human albumin raise the potential of allergic and/or immune responses in subjects being treated with the albumin composition. See e.g., Bosse et al., J Clin. Pharmacol. 45:57-67 (2005). Further, non-enzymatic glycation of albumin, e.g., glucose binding at Lys525 and Lys548, and the formation of Amadori products at these residues can induce conformational changes in local protein secondary structure, thereby influencing the ligand binding and functional activity of albumin. See e.g., Shaklai et al., J. Biol. Chem. 259(6):3812-17 (1984); Wada, J. Mass. Spectrom. 31:263-266 (1996); Howard et al., J. Biol. Chem. 280(24):22582-89 (2005). Therefore, while not intending to be bound by any particular theory of operation, it is believed that deglycation of albumin, particularly recombinant albumin produced in yeast, may yield albumin having advantageous tolerability and stability with respect to conjugates formed therewith. Accordingly, in particular embodiments of the invention, the recombinant albumin may be deglycated prior to proceeding with the conjugation reaction.

Generally, deglycation of albumin may be carried out using any technique and under any conditions known to those of skill in the art to be useful for the reduction of non-enzymatically glycated proteins. Exemplary methods are described by Miksik et al., J. Chromatogr. B. Biomed. Sci. Appl. 699(1-2):311-45 (1997), the contents of which are hereby incorporated by reference in their entirety. In some embodiments, non-enzymatically glycated albumin may be reduced by chromatographic methods. In certain embodiments, the chromatography can be any chromatography known to those of skill in the art to be useful for the separation of glycated proteins from nonglycated proteins. By way of example and not by limitation, the chromatography can be size exclusion chromatography, ion exchange chromatography, or affinity chromatography.

In some embodiments, separation of glycated and nonglycated albumin is carried out by size exclusion chromatography. In certain embodiments, any size exclusion gel capable of separating glycated albumin from nonglycated albumin may be used according to the judgment of one of skill in the art. For example, size exclusion chromatography may be carried out with Superose® 6 HR (GE Healthcare, Piscataway, N.J.) equilibrated in, for example 0.05 M phosphate, 0.15 M sodium chloride, pH 6.8. In some embodiments, elution may be carried out in the equilibration buffer at a flow rate of about 0.5 ml/min.

In certain embodiments, size exclusion chromatography may be carried out with Sepharose® CL-4B (Sigma-Aldrich, St. Louis, Mo.) equilibrated in, for example, 0.01 M phosphate buffer, pH 7.2. In some embodiments, elution is carried out in the equilibration buffer at a flow rate of about 20 ml/h. In certain embodiments, individual fractions are dialyzed against, e.g., saturated ammonium sulfate and the precipitate is re-dissolved in 0.01 M phosphate buffer, pH 7.2.

In another embodiment, separation of glycated and nonglycated albumin is carried out by ion exchange chromatography. In certain embodiments, any ion exchange resin capable of separating glycated albumin from nonglycated albumin according to the judgment of one of skill in the art may be used. For example, the ion exchanger may be a strongly basic anion exchanger such as Hydropore AX (Rainin, Woburn, Mass.) equilibrated in, for example, 10 mM phosphate buffer, pH 7.1. In some embodiments, after loading and binding to the resin, elution of albumin is carried out by applying an increasing salt gradient, either linear or stepwise, or a combination thereof. For instance, glycated and nonglycated albumin species may be separated and eluted by contact with a solution comprising 0 to 1 M NaCl, pH 7.1. In other embodiments, the ion exchanger may be a weakly basic anion exchanger such as DEAE Sephacel (GE Healthcare, Piscataway, N.J.) equilibrated in, for example 0.01 M phosphate, pH 7.2. In some embodiments, elution is carried out at 4° C. by an increasing linear gradient of NaCl from 0 to 0.5 M.

In preferred embodiments, the deglycation is carried out by affinity chromatography. Any affinity ligand capable of separating glycated albumin from nonglycated albumin according to the judgment of one of skill in the art may be used. While not intending to be bound by any particular theory, it is believed that recombinant albumin secreted from yeast into a glucose-rich culture medium leads to covalent binding of glucose at lysine residues of albumin. Accordingly, the separation of glycated albumin from non-glycated albumin, wherein the glycated albumin is comprised of covalently bound glucose, may be carried out using boronate affinity chromatography. In certain embodiments, aminophenylboronated agarose serves as the affinity ligand. In certain embodiments, the resin is equilibrated with buffer containing 0.25 M ammonium acetate, 0.05 M magnesium chloride, pH 8.5. Following loading of the albumin sample and binding of glycated species to the resin, elution of non-glycated species may be carried out with the equilibration buffer. Bound glycated proteins may be eluted by contacting the aminophenylboronated agarose resin with 0.1 M Tris-HCl buffer containing 0.2 M sorbitol, pH 8.5. After the majority of bound proteins are eluted, 0.5% acetic acid may be used to regenerate the column and to elute more tightly bound protein species. An exemplary separation of glycated from non-glycated albumin under these conditions is provided in Example 6 below.

In another preferred embodiment, deglycation of albumin by affinity chromatography is carried out using Concanavalin A (Con A) as the affinity ligand. Concanavalin A specifically binds to internal and nonreducing terminal alpha-mannosyl groups of various sugars. Under certain conditions, Con A may selectively bind glycated albumin species, where the sugar(s) in question are those other than glucose, such as mannose, galactose, lactose, and the like. Furthermore, Con A may successfully bind to albumin species composed of more complex, i.e., higher-order sugars which are O-linked to the recombinant albumin via covalent bonds onto the side-chain oxygen atoms found in amino-acid residues such as serine and/or threonine. In some embodiments, the Con A resin is equilibrated with a solution containing 0.1 M acetate buffer, 1M NaCl, 1 mM MgCl₂, 1 mM MnCl₂, 1 mM CaCl₂, pH 6. Following loading of the albumin sample and binding of glycated species to the resin, non-glycated albumin species are eluted immediately in equilibration buffer, while elution of the glycated species may be carried out with 0.1 M glucose, 0.1 M mannose in equilibration buffer. An exemplary separation of glycated from non-glycated albumin under these conditions is provided in Example 7 below.

In certain embodiments, eluates containing deglycated albumin may be filtered with a low molecular weight filter to concentrate the sample and wash away salts. In some embodiments, ultrafiltration may be carried out with an Amicon® 10 kDa Millipore filter (Millipore Corporation, Bedford, Mass.). In certain embodiments, the recombinant albumin may be washed with sterile water. In other embodiments the recombinant albumin may be washed with 0.9% saline (154 mM NaCl). In other embodiments the recombinant albumin may be washed with sterile buffer.

In certain embodiments, the albumin solution may be concentrated to about 5-250 mg/ml of total protein, corresponding to about 0.5-25% albumin. In some embodiments, the final concentration of the albumin solution comprises about 5 mg/ml, about 10 mg/ml, about 20 mg/ml, about 40 mg/ml, about 80 mg/ml, about 120 mg/ml, about 150 mg/ml, about 175 mg/ml, about 200 mg/ml, about 225 mg/ml, or about 250 mg/ml total protein. In some embodiments, the albumin solution comprises about 0.5%, about 1%, about 2%, about 4%, about 8%, about 12%, about 15%, about 17.5%, about 20%, or about 25% albumin. The albumin sample may then be reformulated in a desired formulation composition.

Determination of the efficiency of deglycation may be performed according to any method known in the art for the measurement of glycated proteins. In some embodiments, the deglycation efficiency may be determined by any assays known in the art useful for measuring glycated albumin. In some embodiments, the measurement of glycated albumin is carried out by a fructosamine, assay as described in U.S. Pat. No. 5,866,352, the contents of which are hereby incorporated by reference in its entirety. Fructosamine is formed due to a non-enzymatic Maillard reaction between glucose and amino acid residues of proteins. In some embodiments, measurement of glycated albumin is carried out by the nitroblue tetrazolium (NBT) colorimetric method, as described by Mashiba et al., Clin. Chim. Acta 212:3-15 (1992). This method is based on the principle of NBT reduction by the ketoamine moiety of glycated proteins in an alkaline solution. In some embodiments, the measurement of glycated albumin is carried out by an enzyme-linked boronate immunoassay (ELBIA) as described by Ikeda et al., Clin. Chem. 44(2):256-63 (1998). This method depends on the interaction of boronic acids and cis-diols of glycated albumin trapped by anti-albumin antibodies coated onto a microtiter plate well.

### 5.7.6 Deglycosylation of Albumin

In another embodiment, deglycosylation of albumin may be carried out by enzymatic methods. The enzyme can be any enzyme known to those of skill in the art that is capable of removing sugars from proteins. In some embodiments, the enzyme is an endoglycosidase. In some embodiments, the enzyme is endoglycosidase D. In some embodiments, the enzyme is endoglycosidase H. In some embodiments, the enzyme is endoglycosidase F. In some embodiments, deglycation of albumin is carried out by contacting the albumin with a plurality of endoglycosidases. Generally, the glycated albumin is contacted with the deglycating enzyme under conditions suitable for removal of sugars known to those of skill in the art. Such conditions include, for example, contacting the glycated albumin with the enzyme in suitable pH, at suitable enzyme concentration, at a suitable temperature and for a suitable time. In certain embodiments, enzymatic deglycosylation may be combined, i.e., followed with the chromatographic deglycation steps as described supra.

### 5.7.7 Blocking Non-Cys34 Reactive Sites of Albumin

If desired, the recombinant albumin may be further processed for favorable specificity of conjugation, i.e. to reduce the likelihood of formation of non-Cys34 conjugates. In a preferred embodiment, a single compound comprising a therapeutic group and a reactive group, preferably a maleimide group, covalently binds to a single defined site of albumin, or a fragment, variant, or derivative thereof. In a particularly preferred embodiment, the single site of binding to albumin is the thiol group of Cys34. Accordingly, in certain embodiments, the formation of non-Cys34 albumin conjugates may be reduced by blocking other potential reactive sites on albumin.

In some embodiments, the recombinant albumin may be contacted with agents which chemically block residues at which covalent adduct formation is known to occur on human serum albumin. Any agent known in the art capable of blocking reactive sites on albumin other than Cys34 may be used. In some embodiments, the agent blocks a lysine residue. Albumin contains 52 lysine residues, 25-30 of which are located on the surface of albumin and may be accessible for conjugation. Accordingly, in some embodiments, the agent blocks any lysine residue of albumin known to those of skill in the art as having the potential to form covalent adducts. In some embodiments, the compound blocks Lys71 of albumin. In some embodiments, the compound blocks Lys 199 of albumin. In some embodiments, the agent blocks Lys351 of albumin. In some embodiments, the agent blocks Lys525 of albumin. In some embodiments, the agent blocks Lys541 of albumin.

In certain embodiments, non-Cys34 reactive sites on albumin are blocked by contact with a non-steroidal anti-inflammatory drug (NSAID). In some embodiments, non-Cys34 reactive sites on albumin are blocked by contact with acetylsalicylic acid. In some embodiments, the recombinant albumin is contacted with acetylsalicylic acid under conditions sufficient to acetylate Lys71 of albumin. See, e.g., Gambhir et al., J. Bio. Chem. 250(17):6711-19 (1975). In some embodiments, the recombinant albumin is contacted with acetylsalicylic acid under conditions sufficient to acetylate Lys199 of albumin. See, e.g., Walker, FEBS Lett. 66(2):173-5 (1976).

In some embodiments, non-Cys34 reactive sites on albumin are blocked by contact with naproxen acyl coenzyme A (naproxen-CoA). In some embodiments, the recombinant albumin is contacted with naproxen-CoA under conditions sufficient to acylate albumin Lys199, Lys351, or Lys541, or a combination thereof. See, e.g., Olsen et al., Anal. Biochem. 312(2):148-56 (2003).

In a more preferred embodiment, non-Cys34 reactive sites on albumin are blocked by contact with molecules having a high affinity for certain sites on albumin's surface, yet do not form covalent adducts onto albumin's surface. In some embodiments, non-Cys34 reactive sites are rendered less reactive, i.e. less nucleophilic by formulating either serum albumin or recombinant albumin in a buffer which assists in limiting non-Cys34 reactivities, for example, by using a buffer of lower pH rather than neutral pH , i.e., 3<pH<7.

### 5.8 Conjugation of Albumin to a Therapeutic Compound

In another aspect of the invention, the process of forming a conjugate comprises contacting albumin with a compound comprising a therapeutic group and a reactive group, under reaction conditions wherein the reactive group is capable of covalently binding the Cys34 thiol of the albumin to form a conjugate. In some embodiments, the conjugation reaction may proceed in any liquid medium containing albumin.

In some embodiments, the albumin is contacted by the compound in the blood, milk, or urine of a transgenic non-human animal expressing recombinant albumin under conditions sufficient to form a conjugate. In some embodiments, the albumin is contacted by the compound in a crude or clarified lysate of any host cell transformed to produce recombinant albumin, for example an animal cell, a plant cell, a bacterial cell, or a yeast cell, under conditions sufficient to form a conjugate. In some embodiments, the albumin is contacted by the compound in the culture medium of a host organism producing recombinant albumin, wherein the recombinant albumin is secreted therein, under conditions sufficient to form a conjugate. In some embodiments, the albumin is contacted by the compound in a purified albumin solution, for instance a solution resulting from purification by any of the chromatographic methods, or a combination thereof, described supra, under conditions sufficient to form a conjugate. In some embodiments, the albumin is contacted by the compound in a serum albumin solution.

In some embodiments, the albumin is contacted by the compound in a purified albumin solution, wherein the albumin is enriched for mercaptalbumin, under conditions sufficient to form a conjugate. In some embodiments, the albumin is contacted by the compound in a purified albumin solution, wherein the albumin is deglycated, under conditions sufficient to form a conjugate. In some embodiments, the albumin is contacted by the compound in a purified albumin solution, wherein the non-Cys34 reactive sites of albumin have been covalently or non-covalently blocked, under conditions sufficient to form a conjugate. In some embodiments, the albumin is contacted by the compound in a purified albumin solution, wherein the albumin is enriched for mercaptalbumin and deglycated, under conditions sufficient to form a conjugate. In some embodiments, the albumin is contacted by the compound in a purified albumin solution, wherein the albumin is enriched for mercaptalbumin, and the non-Cys34 reactive sites have been covalently or non-covalently blocked, under conditions sufficient to form a conjugate. In some embodiments, the albumin is contacted by the compound in a purified albumin solution, wherein the albumin is deglycated, and the non-Cys34 reactive sites have been covalently or non-covalently blocked, under conditions sufficient to form a conjugate. In some embodiments, the albumin is contacted by the compound in a purified albumin solution, wherein the albumin is enriched for mercaptalbumin, deglycated, and the non-Cys34 reactive sites have been covalently or non-covalently blocked, under conditions sufficient to form a conjugate.

Generally, reaction conditions which favor the covalent binding of the Cys34 thiol of recombinant albumin to the reactive group of the compound will include a suitable pH. While not intending to be bound by any particular theory, it is believed that human serum albumin unfolds and denatures into an elongated random coil at a pH below 3.0. Accordingly, in certain embodiments, the recombinant albumin is contacted with the compound at a pH of at least 3.0. In some embodiments, the recombinant albumin is contacted with the compound at a low to neutral pH. In particular embodiments, the pH is between about 4.0 and 7.0. In some embodiments, the pH is between 4.0 and 5.0. In some embodiments, the pH is between about 5.0 and 6.0. In some embodiments, the pH is between about 6.0 and 7.0. In some embodiments, the pH is about 3.0, 3.5, 4.0, 4.5, 5.0, 5.5, 6.0, 6.5, or 7.0.

Favorable reaction conditions leading to the formation of a conjugate will also include a suitable temperature. A suitable temperature for conjugation will vary depending on the relative purity of the recombinant albumin preparation. In particular embodiments, where the recombinant albumin is contacted by the compound in a culture medium, with or without the host organism, or in a crude or clarified lysate of the host organism, the reaction may be carried out at about 34-40° C., about 35-39° C., or about 36-38° C. In a particular embodiment the recombinant albumin is contacted by the compound at about 37° C. In other embodiments, where the conjugation reaction proceeds in a purified recombinant albumin solution, for instance a recombinant albumin solution resulting from purification by any of the chromatographic methods, or a combination thereof, described supra, the reaction may be carried out at about 17-25° C., about 18-24° C., or about 19-23° C. In some embodiments, the reaction is carried out at about 20-25° C. In a particular embodiment, where the conjugation reaction proceeds in a purified albumin solution, the reaction is carried out at about 20-25° C. and no higher. In another embodiment, reaction may be performed under cold conditions, e.g., about +1° C.-+8° C. The reaction may be slower than at higher temperatures, yet may yield a albumin conjugate product that is more specific to Cys34.

Favorable reaction conditions leading to the formation of a conjugate will also include a suitable reaction time. In certain embodiments, the recombinant albumin is contacted with the compound for at least 1, 2, 3, 4, 5, 6, 7, 8, 9, 10, 15, 20, 25, 30, 35, 40, 45, 50, 55 or 60 minutes. In a particular embodiment, the recombinant albumin is contacted with the compound for at least 30 minutes. In some embodiments, the recombinant albumin is contacted with the compound for about 1-60 minutes, about 5-55 minutes, about 10-50 minutes, about 20-40 minutes, or about 25-35 minutes.

In other embodiments, the recombinant albumin is contacted with the compound for at least 0.1, 1, 2, 3, 4, 5, 6, 7, 8, 9, 10, 11, 12, 13, 14, 15, 16, 17, 18, 19, 20, 21, 22, 23, or 24 hours. In some embodiments, the recombinant albumin is contacted with the compound for at least 1, 2, 3, 4, 5, 6, 7, 8, 9, 10, 15, or 20 days.

Favorable reaction conditions leading to the formation of a conjugate will also include a suitable stoichiometry of reactants in solution. The titer of albumin in solution may be determined according to any method known in the art, for example SDS-PAGE; albumin specific enzyme linked immunoassay (ELISA); absorbance based assays (280 nm, 205 nm); colorimetric assays, such as Lowry assay, Bradford assay, Bicinchoninic assay; Kjeldahl method, and the like. Generally, the final molar ratio of compound to albumin will vary, depending on the relative purity of the solution in which a compound is contacted with albumin, as well as the purity of the albumin to which contact is made. For instance, where the compound is added to a solution containing intact or lysed host cells, host proteins and antigens may compete with recombinant albumin for binding to the reactive group of the compound, thus requiring a higher molar amount of compound relative to albumin. In other embodiments, where the compound is added to a purified preparation of albumin, e.g., albumin which is uncapped, deglycated, and/or blocked at non-Cys34 reactive sites, a lower molar amount of compound relative to albumin may be required. Thus, in some embodiments, the conjugation reaction may comprise a solution containing a higher molar concentration of compound relative to albumin. In some embodiments, the conjugation reaction comprises a solution containing an equimolar concentration of compound to albumin. In particular embodiments, the conjugation reaction comprises a solution containing a lower molar concentration of compound to albumin.

In some embodiments, the albumin is contacted with a compound in a solution comprising a final molar ratio of compound to albumin of about 0.1:1 to about 10,000:1. In some embodiments, the final molar ratio is about 7500:1, 5000:1, about 2500:1, about 1000:1, about 750:1, about 500:1, about 250:1, about 100:1, about 75:1, about 50:1, about 25:1, about 10:1, about 7.5:1, about 5:1, about 2.5:1, or about 1:1.

In some embodiments, the final molar ratio is between about 0.1:1 to 1:1. In some embodiments, the final molar ratio is about 0.1:1, 0.2:1, 0.3:1, 0.4:1, 0.5:1, 0.6:1, 0.7:1, 0.8:1, 0.9:1. In a particular embodiment, the final molar ratio of compound to albumin is about 0.7:1.

In particular embodiments, where the compound is formulated in a powder form, the compound may be solubilized using sterile water prior to addition to the conjugation reaction. In other embodiments, the compound may be solubilized in aqueous buffer, preferably set at a pH no higher than 9.0. In a preferred embodiment, the solubilized compound is contacted with the albumin by dropwise addition of the compound to the albumin solution, under conditions sufficient to form a conjugate.

### 5.9 Purification of Conjugates

Solutions comprising conjugates formed according to the processes described herein may be purified to separate monomeric forms of the conjugate from host proteins, antigens, endotoxins, particulate matter, reducing agents, modifying enzymes, salts, unbound compound, unbound albumin, either capped or uncapped, or monomeric or dimeric, and/or aggregate forms of the conjugate according to the steps described below.

Thus, in some embodiments, a solution comprising conjugates formed in a culture medium containing the host organism, wherein recombinant albumin was secreted by the host organism, may be purified according to the steps below. In some embodiments, a solution comprising conjugates formed in a culture supernatant wherein the recombinant albumin was secreted by a host organism, and the host organism was separated from the culture medium prior to conjugation, may be purified according to the steps below. In some embodiments, a solution comprising conjugates formed in a clarified lysate wherein the recombinant albumin was produced intracellularly, and the host organism was lysed and separated from the culture medium prior to conjugation, may be purified according to the steps below.

In some embodiments, a solution comprising conjugates formed in a purified solution of recombinant albumin produced from a host cell, may be purified according to the steps below. In some embodiments, conjugates formed in a purified solution of recombinant albumin produced from a host cell, wherein the albumin is enriched for mercaptalbumin, may be purified according to the steps below. In some embodiments, conjugates formed in a purified solution of recombinant albumin produced from a host cell, wherein the albumin is deglycated, may be purified according to the steps below. In some embodiments, conjugates formed in a purified solution of recombinant albumin produced from a host cell, wherein the albumin is blocked at non-Cys34 reactive sites, may be purified according to the steps below.

In some embodiments, conjugates formed in a purified solution of recombinant albumin produced from a host cell, wherein the albumin is enriched for mercaptalbumin and deglycated, may be purified according to the steps below. In some embodiments, conjugates formed in a purified solution of recombinant albumin produced from a host cell, wherein the albumin is deglycated and blocked at non-Cys34 reactive sites, may be purified according to the steps below. In some embodiments, conjugates formed in a purified solution of recombinant albumin produced from a host cell, wherein the albumin is enriched for mercaptalbumin and blocked at non-Cys34 reactive sites, may be purified according to the steps below. In some embodiments, conjugates formed in a purified solution of recombinant albumin produced from a host cell, wherein the albumin is enriched for mercaptlabumin, deglycated, and blocked at non-Cys34 reactive sites, may be purified according to the steps below.

In preferred embodiments, conjugation products may be purified by hydrophobic interaction chromatography. In some embodiments, any hydrophobic resin capable of binding albumin according to the judgment of one of skill in the art may be used. In some embodiments, the hydrophobic resin can be octyl sepharose, butyl sepharose, or phenyl sepharose, or a combination thereof. In preferred embodiments, the purification comprises a 2-step purification, optionally followed by ultrafiltration.

In some embodiments, HIC purification of the conjugate comprises a first flow through step with phenyl sepharose to remove unbound compound from solution. In particular embodiments, this flow through step occurs immediately after the conjugation reaction to limit the formation of non-Cys34 albumin conjugates. Phenyl sepharose resin may be equilibrated in low salt, for example 5 mM ammonium sulfate, 5 mM magnesium sulfate or 5 mM ammonium phosphate in a buffer of 5 mM sodium octanoate, set at neutral pH (e.g. Phosphate buffer pH 7.0). In some embodiments, conductivity of the equilibration buffer is set at 5.8 mS/cm. Under these conditions, unconjugated compound binds to the resin, while the majority of compound-albumin conjugate flows through, and may be eluted within 5-6 column volumes.

Following elution from the phenyl sepharose column, the flow through may be optionally subjected to a mild degradation step to further reduce the amount of non-Cys34 albumin conjugation products. The degradation may be accomplished by incubating the flow through at room temperature and neutral pH for up to 7 days before proceeding further with purification. In some embodiments, the phenyl sepharose flow through may be incubated for 1, 2, 3, 4, 5, 6, or 7 days at room temperature prior to proceeding with the second hydrophobic interaction chromatography step. In some embodiments, the phenyl sepharose flow through is incubated for 1 day at room temperature. In some embodiments, the phenyl sepharose flow through is incubated for 2 days at room temperature. In some embodiments, the phenyl sepharose flow through is incubated for 3 days at room temperature. In some embodiments, the phenyl sepharose flow through is incubated for 4 days at room temperature. In some embodiments, the phenyl sepharose flow through is incubated for 5 days at room temperature. In some embodiments, the phenyl sepharose flow through is incubated for 6 days at room temperature. In some embodiments, the phenyl sepharose flow through is incubated at neutral pH for 7 days room temperature.

In particular embodiments, following the mild degradation step, the phenyl sepharose flow through may be subjected to a second phenyl sepharose flow through step, under identical conditions as the first, e.g., 5 mM ammonium sulfate, 5 mM magnesium sulfate or 5 mM ammonium phosphate in a buffer of 5 mM sodium octanoate, pH 7.0; conductivity of 5.8 mS/cm, to remove unconjugated compound molecules resulting from the degradation step.

Following phenyl sepharose chromatography, the flow through is then applied to a second hydrophobic interaction chromatography comprising contact with butyl sepharose resin. Methods for the purification of albumin conjugates using butyl sepharose hydrophobic interaction chromatography are described in U.S. patent application Ser. No. 11/112,277, the contents of which are incorporated by reference in its entirety. This purification step separates monomeric compound-albumin conjugates from free unbound albumin, dimeric albumin, additional unbound compound, and aggregate forms of conjugate. In some embodiments, butyl sepharose resin may be equilibrated in 750 mM ammonium sulfate, 5 mM sodium octanoate, set at neutral pH (e.g. Phosphate buffer pH 7.0). Following loading and binding to the resin, separation of monomeric compound-albumin conjugates may be achieved by applying a decreasing salt gradient, either linear or stepwise, or a combination thereof. For example, monomeric compound-albumin conjugates may be eluted by contact with a solution comprising 0-750 mM (NH₄)2SO₄.

In some embodiments, non-conjugated albumin may be eluted by contact with a solution comprising about 750 mM (NH₄)₂SO₄, at a conductivity of 118 mS/cm. In some embodiments, dimeric non-conjugated albumin may be eluted by contact with a solution comprising about 550 mM (NH₄)₂SO₄, at a conductivity of 89 mS/cm.

In some embodiments, monomeric conjugated albumin may be eluted by contact with a solution comprising about 50 to 150 mM (NH₄)₂SO₄. In some embodiments, monomeric conjugated albumin may be eluted by contact with a solution comprising about 75 to 125 mM (NH₄)₂SO₄. In some embodiments, monomeric conjugated albumin may be eluted by contact with a solution comprising about 100 mM (NH₄)₂SO₄, at a conductivity of 21 mS/cm.

In some embodiments, the conjugate may be desalted and concentrated by ultrafiltration following HIC purification, for instance by using an Amicon® ultra centrifugal (30 kDa) filter device (Millipore Corporation, Bedford, Mass.). In some embodiments, the conjugate may be reformulated in a desired formulation composition. In other embodiments, the conjugate is prepared for long term storage by immersing the conjugate solution in liquid nitrogen and lyophilizing the conjugate and storing the conjugate at −20° C.

### 6. EXAMPLES

The invention is illustrated by the following examples which are not intended to be limiting in any way. The chromatographic methods of the following examples were performed using an AKTA purifier (Amersham Biosciences, Uppsala, Sweden).

### 6.1 Example 1

### Purification of Recombinant Albumin Expressed in Pichia pastoris

This example demonstrates purification by various chromatographic methods of recombinant albumin expressed in Pichia pastoris. Recombinant albumin was expressed using the Pichia Expression Kit (Invitrogen, Carlsbad, Calif.) according to manufacturer's protocol.

### 6.1.1 DEAE Sepharose: Weak Anion Exchange Chromatography

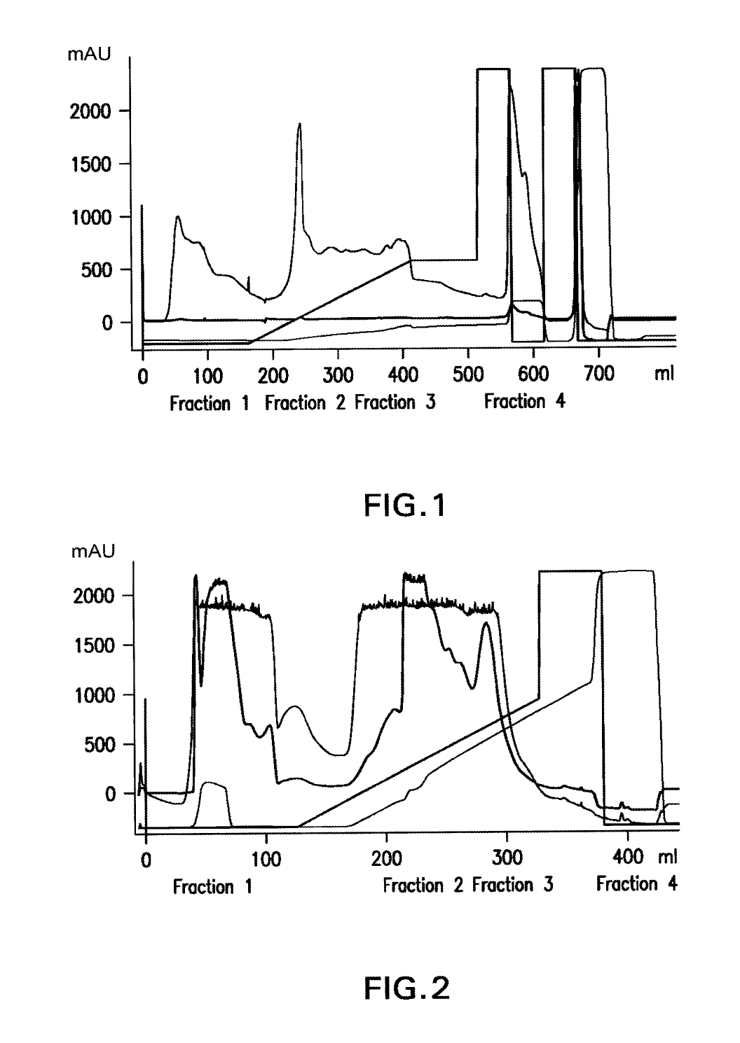

Purification of recombinant human albumin expressed in Pichia pastoris was performed on a column of DEAE sepharose equilibrated in 10 mM sodium phosphate buffer, pH 7.0. A decreasing salt gradient was applied as follows (50 ml column volume, 2 ml/min flow rate): 66 mM sodium phosphate over 5 column volumes; 66 mM sodium phosphate over 2 column volumes; 200 mM sodium phosphate over 0 column volumes; 200 mM sodium phosphate over 1 column volume; regeneration in 20 mM Tris-HCl buffer and 2M NaCl, pH 8.0. In FIG. 1 the purified albumin fraction elutes during the increasing sodium phosphate gradient as fraction.

### 6.1.2 0 Sepharose: Strong Anion Exchange Chromatography

Purification of recombinant human albumin expressed in Pichia pastoris was performed on a column of Q sepharose equilibrated in 20 mM Tris HCl buffer, pH 8.0. An increasing salt gradient was applied as follows (50 ml column volume, 2.5 ml/min flow rate): 1 M NaCl over 8 column volumes; 2 M NaCl over 0 column volumes; 2 M NaCl over 2 column volumes. In FIG. 2 the purified albumin fraction elutes during the increasing NaCl gradient from 0 to 1 M NaCl.

### 6.1.3 Hitrap Blue: Affinity Chromatography

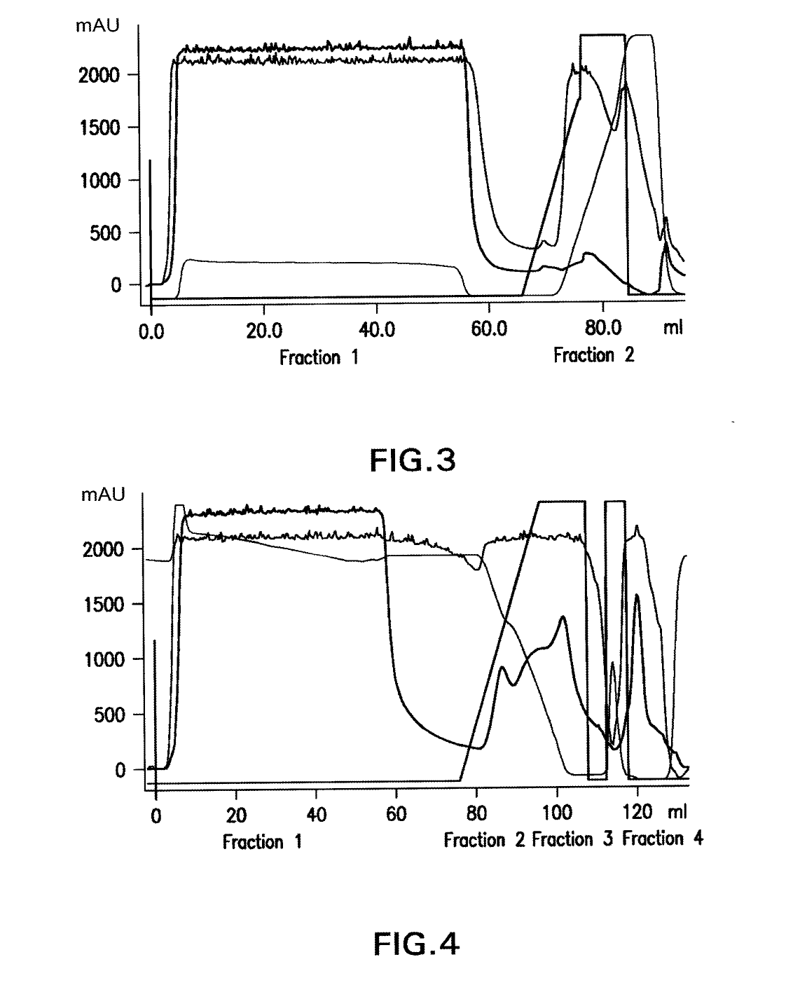

Purification of recombinant human albumin expressed in Pichia pastoris was performed on a HiTrap™ Blue HP (GE Healthcare, Piscataway, N.J.) column equilibrated in 20 mM Tris HCl buffer, pH 8.0. An increasing salt gradient was applied as follows (5 ml column volume, 2.5 ml/min flow rate): 1 M NaCl over 2 column volumes; 2 M NaCl over 0 column volumes; 2 M NaCl over 1 column volume. In FIG. 3 the purified albumin fraction elutes during the increasing NaCl gradient from 0 to 2 M NaCl.

### 6.1.4 Phenyl Sepharose: Hydrophobic Interaction Chromatography

Purification of recombinant human albumin expressed in Pichia pastoris was performed on a column containing phenyl sepharose equilibrated in 20 mM sodium phosphate, 5 mM sodium caprylate and 750 mM (NH₄)₂SO₄, pH 7.0. An decreasing salt gradient was applied as follows (5 ml column volume, 5 ml/min flow rate): 20 mM sodium phosphate, 5 mM sodium caprylate over 2 column volumes; wash performed with water over 1 column volume; 20% ethanol over 1 column volume; and water over 1 column volume. In FIG. 4 the purified albumin fraction elutes during the decreasing gradient from 750 to 0 M (NH₄)₂SO₄.

### 6.2 Example 2

### Purification of Recombinant Albumin Following Enrichment of Mercaptalbumin

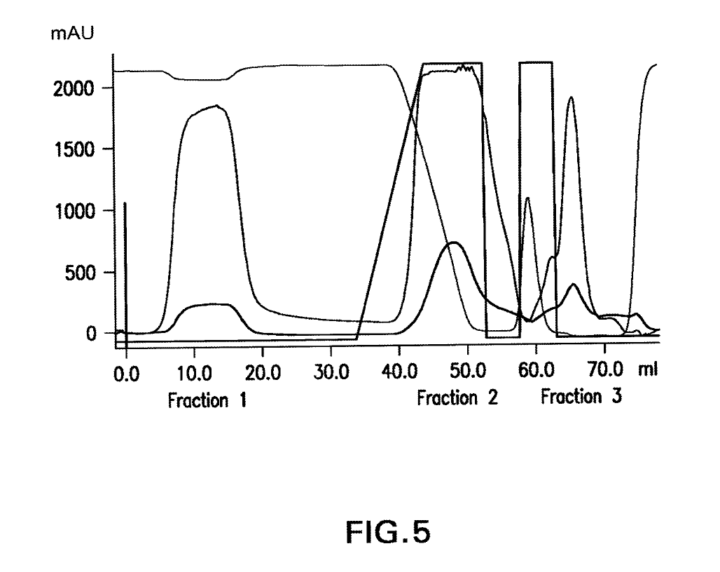

This example demonstrates purification by phenyl sepharose hydrophobic interaction chromatography of recombinant albumin expressed in Pichia pastoris and enriched for mercaptalbumin. Recombinant albumin (0.2% final) was treated with 74 mM thioglycolic acid in 250 mM Tris-acetate buffer for 20 hours at 4° C. Purification was performed on a column containing phenyl sepharose equilibrated in 20 mM sodium phosphate, 5 mM sodium caprylate and 750 mM (NH₄)₂SO₄, pH 7.0. An decreasing salt gradient was applied as follows (5 ml column volume, 5 ml/min flow rate): 20 mM sodium phosphate, 5 mM sodium caprylate over 2 column volumes; wash performed with water over 1 column volume; 20% ethanol over 1 column volume; and water over 1 column volume. In FIG. 5 the purified albumin fraction elutes during the decreasing gradient from 750 to 0 M (NH₄)₂SO₄. The F2 were collected and concentrated with a Amicon 10 kDa Millipore filter and washed with water for injection (WFI) four times.

### 6.3 Example 3

### Purification of Recombinant Albumin Following Deglycation

This example demonstrates deglycation of human serum albumin by affinity chromatography using amino-phenyl boronic acid and concanavalin A as ligands. Chromatography was performed on an AKTA purifier (Amersham Biosciences, Uppsala, Sweden).

### 6.3.1 Amino-Phenyl Boronic Acid Chromatography with Agarose

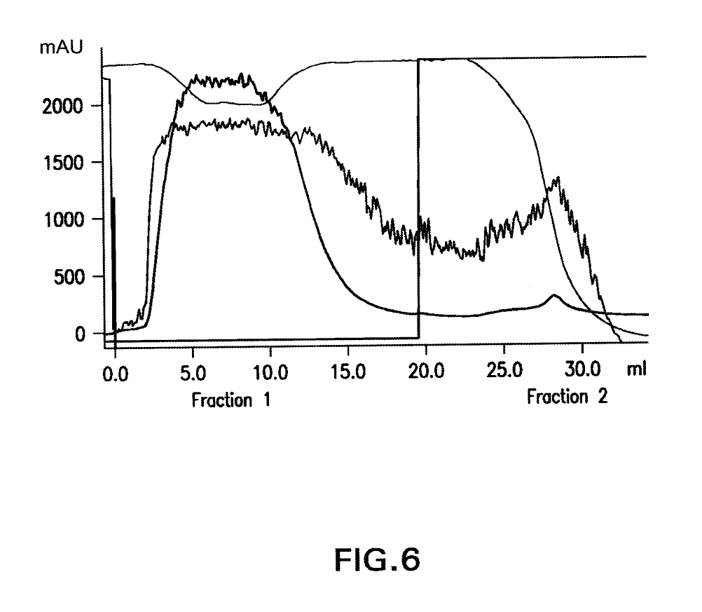

Amino phenyl boronic acid resin with agarose (Sigma, St. Louis, Mo.) was washed and equilibrated with 4 column volumes of 0.25 M ammonium acetate, pH 8.5, 0.05 MgCl₂ (0.5 ml/min flow rate). 25% human serum albumin solution (Cortex Biochem, San Leandro, Calif.) was diluted 1:2 in equilibrating buffer and loaded on the column. The flow through was collected (F3) and the column was washed with 4 column volumes of equilibrating buffer. Elution was performed with 3 column volumes of 0.1 M Tris, pH 8.5 with 0.2 M sorbitol and collected in F2. F3 and F2 were concentrated with a Amicon 10 kDa Millipore filter and washed with water for injection (WFI, Abbott Laboratories, Abbott Park, Ill.) four times. The column was regenerated with 5 column volumes of 0.1 M borate buffer, pH 9.8, 1 M NaCl; 5 column volumes of 0.1 M borate buffer, pH 9.8, 5 column volumes of water, and 5 column volumes of 2 M NaCl. A representative chromatogram is shown in FIG. 6.

### 6.3.2 Concanavalin A (Con A) Chromatography

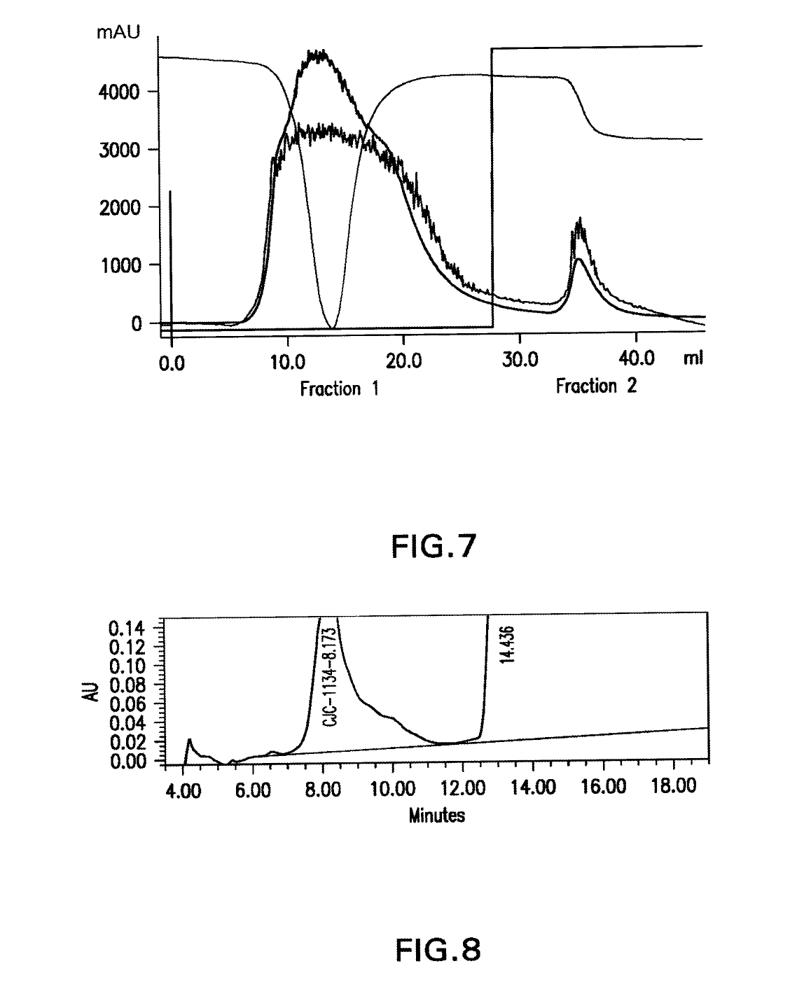

Con A resin (Amersham, Piscataway, N.J.)) was washed and equilibrated with 4 column volumes 0.1 M acetate buffer, pH 6.0, 1 M NaCl 1 mM MgCl2, 1 mM MgCl2, 1 mM CaCl₂ (2 ml/min flow rate). 20% recombinant human serum albumin solution (North China Pharmaceutical Co., Shijiazhuang, China) was diluted 1:2 in equilibrating buffer and loaded on the column. The flow through was collected (F3) and the column was washed with 4 column volumes of equilibrating buffer. Elution was performed with 3 column volumes of equilibration buffer plus 0.1 M glucose and 0.1 M mannose, and collected in F2. F3 and F2 were concentrated with a Amicon 10 kDa Millipore filter and washed with water for injection (WFI, Abbott Laboratories, Abbott Park, Ill.) four times. The column was regenerated with 5 column volumes of 0.1 M borate buffer, pH 9.8; 1 M NaCl; 5 column volumes of water; 5 column volumes of 0.1 M borate buffer, pH 8.5; and 5 column volumes of 0.1 M borate buffer, pH 4.5. A representative chromatogram is shown in FIG. 7.

### 6.4 Example 4

### Purification of Monomeric Compound-Albumin Conjugates

Recombinant albumin expressed in Pichia pastoris was purified and treated with thioglycolic acid as described in Example 2, supra, and purified by phenyl sepharose HIC prior to conjugation with CJC-1134 (Exendin-4 comprising the reactive group MPA). The conjugation reaction comprised 35 μl of 10 mM CJC-1134 combined with 175 μl of mercaptalbumin enriched albumin at a final molar ratio of 0.7:1. The reaction proceeded for 30 minutes at 37° C., and was then stored at 4° C. for liquid chromatography/mass spec analysis and purification by butyl sepharose HIC.

FIG. 8 shows an HPLC chromatogram of unbound CJC-1134 found post conjugation between CJC-1134 and recombinant albumin prior to loading onto a first phenyl sepharose flow through column. Retention time of unbound CJC-1134 is 8.2 minutes, and that of the CJC-1134-albumin conjugate is after 12 minutes.

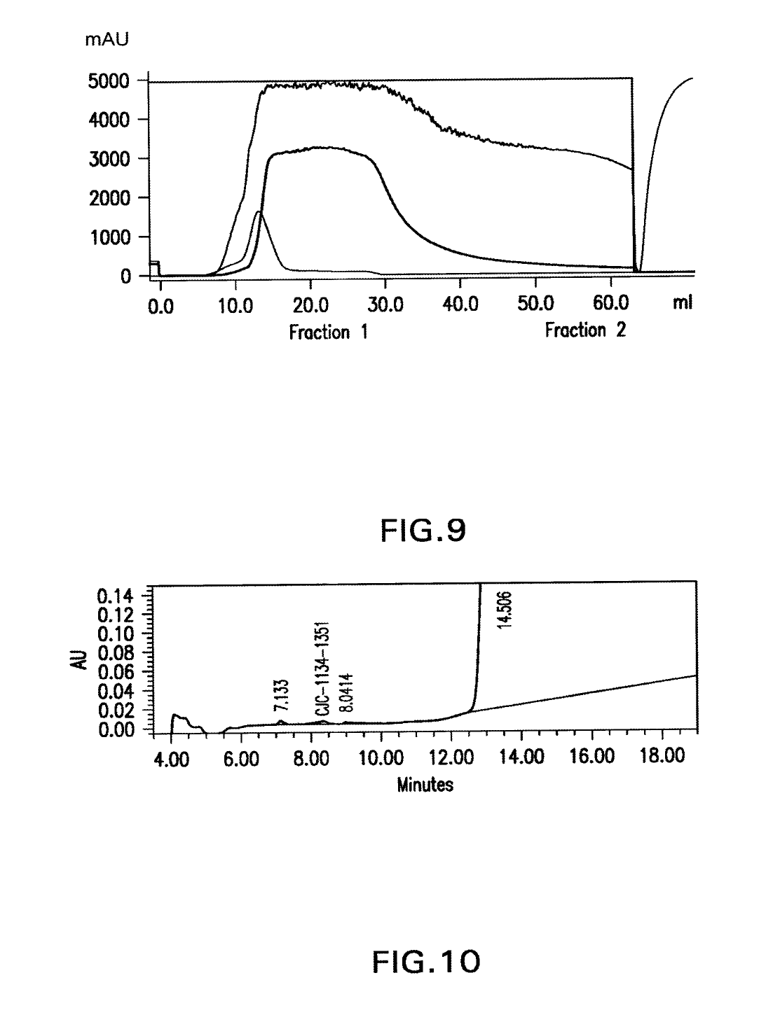

For the first HIC, phenyl sepharose was pre-equilibrated in 20 mM sodium phosphate buffer (pH 7.0) composed of 5 mM sodium octanoate and 5 mM ammonium sulfate. Direct loading of the conjugation reaction onto the resin enabled physical separation of protein (albumin and conjugated albumin) observed in the flow-through from unbound CJC-1134. Therefore, capacity of this resin is reserved primarily for unbound compound comprising a reactive moiety. A representative chromatogram is shown in FIG. 9.

FIG. 10 shows an HPLC chromatogram of unbound CJC-1134 found post conjugation between CJC-1134 and recombinant albumin following loading onto a first phenyl sepharose flow through column. Retention time of unbound CJC-1134 is 8.2 minutes, and that of the CJC-1134-albumin conjugate is after 12 minutes. Thus, the unbound CJC-1134 has been effectively removed from the pool of conjugate reaction products.

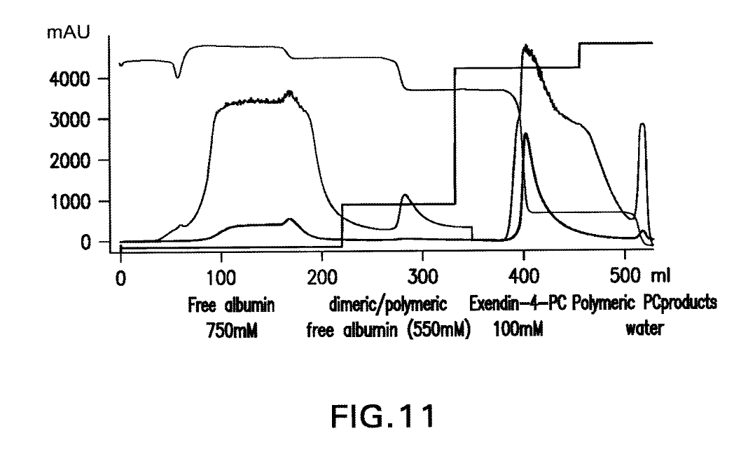

For the second HIC, butyl sepharose resin was equilibrated equilibrated in 20 mM sodium phosphate buffer, 5 mM sodium caprylate, 750 mM (NH₄)₂SO₄, pH 7.0. A decreasing salt gradient was applied as follows (5 ml column volume, 2.5 ml/min flow rate): 20 mM sodium phosphate, 5 mM sodium caprylate, pH 7.0 over 4 column volumes; washed with water for 1 column volume; 20% ethanol over 1 column volume; and water over 1 column volume. The F2 were collected and concentrated with a Amicon 10 kDa Millipore filter and washed with WFI four times. FIG. 11 shows 3 distinct populations eluting at different points along the gradient: about 750 mM (NH₄)₂SO₄, corresponding to non conjugated albumin, about 550 mM (NH₄)₂SO₄, corresponding to dimeric non-conjugated albumin, and about 100 m (NH₄)₂SO₄, corresponding to monomeric conjugated albumin.

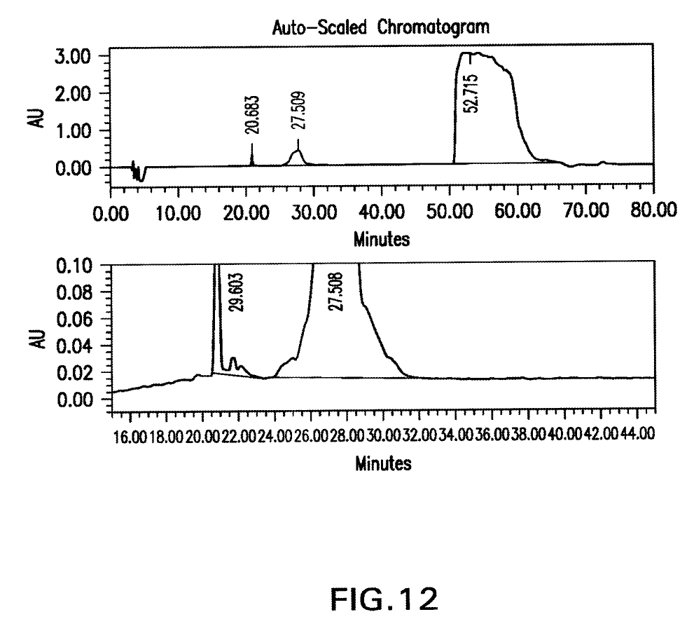

Successful conjugation was also observed between recombinant albumin and a compound comprising GLP-1 and the reactive group MPA. FIG. 12 shows an HPLC chromatogram of unbound DAC-GLP-1 (CJC-1131) found post-conjugation between DAC-GLP-1(CJC-1131) and rHA prior to loading onto a phenyl sepharose flow-through column. Retention time of unbound CJC-1131 is 27.5 min, and that of the albumin conjugate is after 50 min.

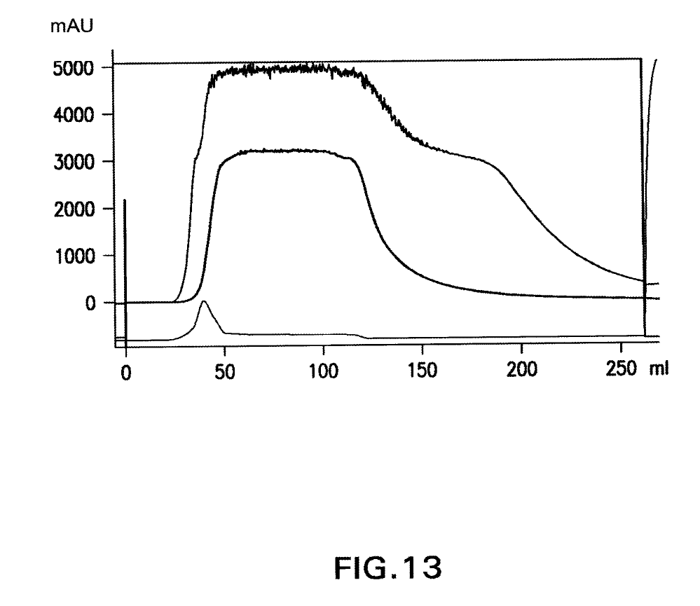

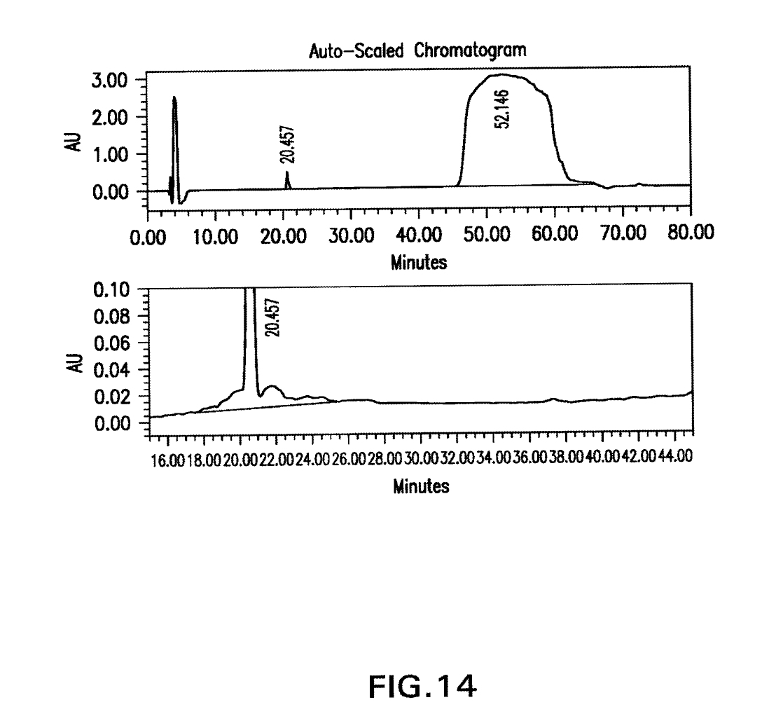

For the first HIC, phenyl sepharose was pre-equilibrated in 20 mM sodium phosphate buffer (pH 7.0) composed of 5 mM sodium octanoate and 5 mM ammonium sulfate. Direct loading of conjugation reaction onto the resin enabled physical separation of protein (albumin and conjugated albumin) observed in flow-through from unbound DAC-GLP-1 (CJC-1131), as shown in FIG. 13. FIG. 14 shows an HPLC chromatogram of unbound DAC-GLP-1 found post-conjugation between DAC-GLP-1 (CJC-1131) and recombinant human albumin following loading of the conjugate reaction onto a phenyl sepharose flow-through column. Retention time of unbound CJC-1131 is 27.5 min, and that of the albumin conjugate is after 46 min. Therefore, unbound CJC-1131 was effectively removed from all protein species. The peak having a retention time of 20.5 min corresponds to octanoate.

GLP-1-albumin conjugates were also prepared for SDS-PAGE and Western Blot analysis. Briefly, following the conjugation reaction described above, about 20 μg of material was diluted in Laemmli 3× buffer, boiled for 3 minutes, and loaded onto an 8% polyacrylamide-bisacrylamide gel. Proteins migrated under non-reducing conditions. Following transfer to nitrocellulose membrane (Constant current; 100 mA/gel for one hour (2 mA/cm2)), membrane staining was performed with Ponceau red and de-stained completely with TBS; membranes were saturated with 0.05% Tween20, 5% milk in Tween20 overnight at 4° C., followed by 3 washes with 0.05% Tween20, in Tween20 for 10 minutes, followed by staining with red Commassie blue and de-stained completely with 30% MeOH, 10% acetic acid. Immunodetection of albumin was performed by incubation with an HRP-labeled goat antibody anti-human albumin (GAHu/Alb/P0, Nordic immunology, batch #5457) for 1 h at room temperature Immunodetection of GLP-1 was performed by 1 hour incubation with a rabbit anti GLP-1 antibody, followed by incubation with an HRP-labeled goat anti-rabbit antibody for 1 hour. Membranes were then washed for 3 washes with TBS-0.05% Tween20 for 10 minutes. Detection of signal was performed with ECL (Amersham Pharmacia Biotech, RPN 2209).

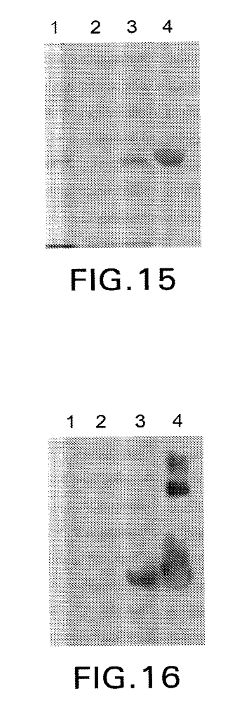

FIG. 15 and FIG. 16 presents a coomassie stain and an anti-albumin Western blot, respectively, of unconjugated recombinant albumin (lane 3), and the reaction products of a GLP-1 albumin conjugation reaction (lane 4). Higher molecular weight species are observed following conjugation relative to unconjugated albumin, reflecting to monomeric and polymeric GLP-1-albumin conjugate species.

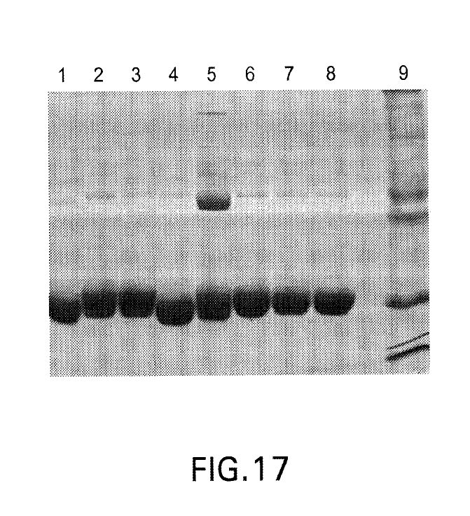

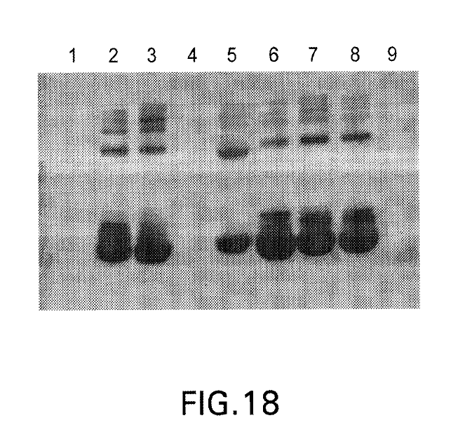

FIG. 17 and FIG. 18 presents a coomassie stain and an anti-GLP-1 Western blot, respectively, of fractions from various stages of purification following a conjugation reaction between GLP-1 and recombinant human albumin, as described above. Samples were loaded as follows:

(1)rHA

(2) Pre-purification

(3) Phenyl F8

(4) Butyl F3 750 mM (NH₄)₂SO₄

(5) Butyl F5 550 mM (NH₄)₂SO₄

(6) Butyl F6A 100 mM (NH₄)₂SO₄ before PC 200-2000 mAU

(7) Butyl F6B 100 mM (NH₄)₂SO₄ PC WFI

(8) Butyl F6B 100 mM (NH₄)₂SO₄ PC Acetate

(9) Standard

### 6.5 Example 4

### Conjugation to Albumin in a Culture Medium

Recombinant human albumin was expressed using the Pichia Expression Kit (Invitrogen, Carlsbad, Calif.) according to manufacturer protocol. Following 3 days of albumin expression and secretion into the culture supernatant at 28-30° C., 100 ml of broth was centrifuged so as to physically separate host cells from crude supernatant. The crude supernatant was then concentrated using Amicon® centrifuge tubes (MW cutoff=10 kDa) to a final protein concentration of 20-100 mg/ml (as estimated using a standardized BCA method), followed by liquid chromatography-electrospray mass spectrometry (LC-EMS) analysis. At day 3, a conjugation reaction was performed at a final molar ratio of 1000×-fold DAC-GLP-1 (CJC-1131) to albumin by direct addition into culture broth composed of host cells.

LC-EMS data prior to and following conjugation reactions indicated that no species corresponding to the MW range of mercaptalbumin was detectable. 1000 ×-fold of CJC-1131 (DAC-GLP-1; Mw=3,721 Da) was added directly into the culture broth (composed of host cells) and allowed to react at 25° C. for 60 min. Following the reaction, host cells were physically separated from crude supernatant using centrifugation. The crude supernatant was then concentrated further using Amicon® centrifugation tubes (Mw cutoff=10 kDa) to a final concentration of 20-100 mg/ml, followed by LC-EMS analysis. A protein species with a total mass of 70,160-70,170 would correspond to the generation of a GLP-1-albumin conjugate. However, no detectable mass of this size was observed following the conjugation reaction.

Conjugation in culture media may be successful where the expression and secretion of recombinant albumin is under conditions where reducing agents, such as L-cysteine, are removed or depleted. Furthermore, since albumin's Cys34 residue may be susceptible to oxidation, the secretion of recombinant albumin may be attempted under more stringent conditions of aeration. By way of example and not by limitation, such fermentation conditions may be favorable for the formation of conjugates in culture media.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1: A process for the preparation of a conjugate, said conjugate comprising albumin covalently linked to a compound, the process comprising purifying the conjugate by a first hydrophobic interaction chromatography followed by a second hydrophobic interaction chromatography.

2: The process of claim 1, wherein the process comprises
(a) subjecting a first solution comprising the conjugate, unconjugated albumin, and unconjugated compound to the first hydrophobic interaction chromatography under conditions wherein said unconjugated compound is separated from said conjugate and unconjugated albumin;
(b) collecting a second solution comprising said conjugate and unconjugated albumin in flow through from said first hydrophobic interaction chromatography;
(c) subjecting said second solution to the second hydrophobic interaction chromatography under conditions wherein said conjugate is separated from said unconjugated albumin; and
(d) collecting a third solution comprising said conjugate, whereby said unconjugated albumin and unconjugated compound have been separated away from said conjugate.

3: The process of claim 1, wherein the first hydrophobic interaction chromatography is phenyl sepharose chromatography.

4: The process of claim 1, wherein the second hydrophobic interaction chromatography is butyl sepharose chromatography.

5: The process of claim 4, wherein the butyl sepharose chromatography comprises:
a. equilibrating butyl sepharose resin in 750 mM ammonium sulfate;
b. contacting the butyl sepharose resin with a solution comprising the conjugate; and
c. applying a decreasing salt gradient from 750-0 mM ammonium sulfate to separate monomeric conjugated albumin species from non-monomeric albumin species.

6: The process of claim 1, wherein the first hydrophobic interaction chromatography is different than the second hydrophobic interaction chromatography.

7: The process of claim 1, wherein the conjugate is formed in a solution by contacting albumin contained in the solution with a compound, said compound comprising a reactive group, under reaction conditions wherein the reactive group is capable of covalently binding cysteine 34 thiol of the albumin to form a conjugate.

8: The process of claim 7, wherein the albumin is mercaptalbumin-enriched albumin.

9: The process of claim 7, wherein the albumin is deglycated albumin.

10: The process of claim 7, wherein the albumin is deglycated albumin enriched for mercaptalbumin.

11: The process of claim 7, wherein said reaction conditions comprise a final molar ratio of the compound to recombinant albumin of 0.1:1 to 1:1.

12: The process of claim 1, wherein the compound comprises an amino acid, a peptide, a protein, an organic molecule, RNA, or DNA.

13: The process of claim 12, wherein the compound comprises a peptide.

14: The process of claim 1, wherein the compound is insulin, atrial natriuretic peptide (ANP), brain natriuretic peptide (BNP), peptide YY (PYY), growth hormone releasing factor (GRF), glucagon-like peptide-1 (GLP-1), exendin-3, or exendin-4.

15: The process of claim 1, wherein the compound comprises a reactive group, wherein the reactive group is a Michael acceptor, a succinimidyl-containing group, a maleimido-containing group or an electrophilic thiol acceptor.

16: The process of claim 15, wherein the reactive group is maleimid-propionic acid (MPA).

17: The process of claim 15, wherein the reactive group is a cysteine residue.

18: The process of claim 7, wherein the albumin is fused to a peptide.

19: The process of claim 18, wherein the peptide is glucagon-like peptide 1, exendin 3, or exendin-4.

20: The process of claim 1, wherein the conjugate is according to the following (SEQ ID NO: 31): 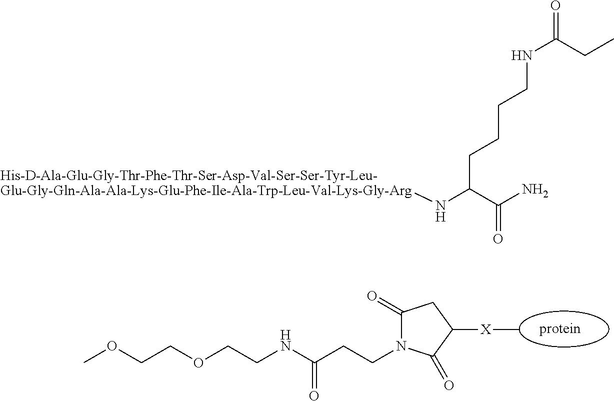
wherein the protein is albumin and X is S of Cysteine 34.

21: The process of claim 1, wherein the conjugate is according to the following (SEQ ID NO. 30): 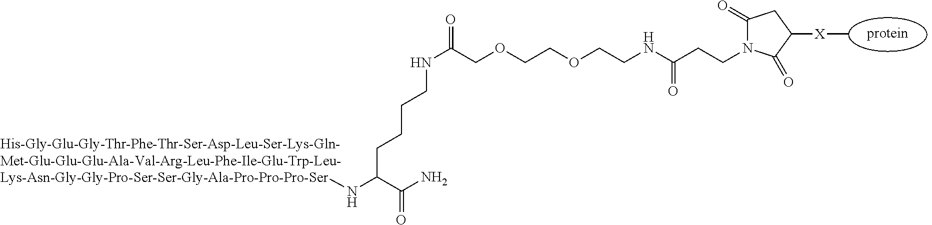
wherein the protein is albumin and X is S of Cysteine 34.

22: The process of claim 3, wherein the phenyl sepharose chromatography comprises:
(a) equilibrating phenyl sepharose resin in a buffer set at neutral pH, comprising 5 mM sodium octanoate, and a salt concentration selected from the group consisting of 5 mM ammonium sulfate, 5 mM magnesium sulfate, and 5 mM ammonium phosphate;
(b) contacting the phenyl sepharose resin with said first solution comprising the conjugate, unconjugated albumin and unconjugated compound; and
(c) collecting the flow-through.

23: The process of claim 1, wherein the first hydrophobic interaction chromatography is phenyl sepharose chromatography, and wherein the second hydrophobic interaction chromatography is butyl sepharose chromatography.

24: The process of claim 23, wherein the phenyl sepharose chromatography comprises:
(a) equilibrating phenyl sepharose resin in a buffer set at neutral pH, comprising 5 mM sodium octanoate, and a salt concentration selected from the group consisting of 5 mM ammonium sulfate, 5 mM magnesium sulfate, and 5 mM ammonium phosphate;
(b) contacting the phenyl sepharose resin with said first solution comprising the conjugate, unconjugated albumin and unconjugated compound; and
(c) collecting the flow-through.

25: The process of claim 23, wherein the butyl sepharose chromatography comprises:
(a) equilibrating butyl sepharose resin in 750 mM ammonium sulfate;
(b) contacting the butyl sepharose resin with the second solution comprising the conjugate and conjugated albumin; and
(c) applying a decreasing salt gradient from 750 to 0 mM ammonium sulfate to separate said conjugate from said unconjugated albumin.

26: The process of claim 24, wherein the butyl sepharose chromatography comprises:
(a) equilibrating butyl sepharose resin in 750 mM ammonium sulfate;
(b) contacting the butyl sepharose resin with the second solution comprising the conjugate and conjugated albumin; and
(c) applying a decreasing salt gradient from 750 to 0 mM ammonium sulfate to separate said conjugate from said unconjugated albumin.

27: The process of claim 22, wherein the buffer is a 20 mM sodium phosphate buffer, pH 7.0.

28: The process of claim 24, wherein the buffer is a 20 mM sodium phosphate buffer, pH 7.0.


SEQUENCE LISTING (34 sequences)

SEQ ID NO: 1 - protein, 37 aa, Artificial Sequence
  Description of Artificial Sequence synthetic peptide
HDEFERHAEGTFTSDVSSYLEGQAAKEFIAWLVKGRG

SEQ ID NO: 2 - protein, 31 aa, Artificial Sequence
  Description of Artificial Sequence synthetic peptide
HAEGTFTSDVSSYLEGQAAKEFIAWLVKGRG

SEQ ID NO: 3 - protein, 20 aa, Artificial Sequence
  VARIANT 17: Xaa = Lys or Arg
SYLEGQAAKEFIAWLVXGRX

SEQ ID NO: 4 - protein, 21 aa, Artificial Sequence
  VARIANT 18: Xaa = Lys or Arg
SSYLEGQAAKEFIAWLVXGRX

SEQ ID NO: 5 - protein, 22 aa, Artificial Sequence
  VARIANT 19: Xaa = Lys or Arg
VSSYLEGQAAKEFIAWLVXGRX

SEQ ID NO: 6 - protein, 23 aa, Artificial Sequence
  VARIANT 20: Xaa = Lys or Arg
DVSSYLEGQAAKEFIAWLVXGRX

SEQ ID NO: 7 - protein, 24 aa, Artificial Sequence
  VARIANT 21: Xaa = Lys or Arg
SDVSSYLEGQAAKEFIAWLVXGRX

SEQ ID NO: 8 - protein, 25 aa, Artificial Sequence
  VARIANT 22: Xaa = Lys or Arg
TSDVSSYLEGQAAKEFIAWLVXGRX

SEQ ID NO: 9 - protein, 26 aa, Artificial Sequence
  VARIANT 23: Xaa = Lys or Arg
FTSDVSSYLEGQAAKEFIAWLVXGRX

SEQ ID NO: 10 - protein, 27 aa, Artificial Sequence
  VARIANT 24: Xaa = Lys or Arg
TFTSDVSSYLEGQAAKEFIAWLVXGRX

SEQ ID NO: 11 - protein, 28 aa, Artificial Sequence
  VARIANT 25: Xaa = Lys or Arg
GTFTSDVSSYLEGQAAKEFIAWLVXGRX

SEQ ID NO: 12 - protein, 29 aa, Artificial Sequence
  VARIANT 26: Xaa = Lys or Arg
EGTFTSDVSSYLEGQAAKEFIAWLVXGRX

SEQ ID NO: 13 - protein, 30 aa, Artificial Sequence
  VARIANT 27: Xaa = Lys or Arg
AEGTFTSDVSSYLEGQAAKEFIAWLVXGRX

SEQ ID NO: 14 - protein, 31 aa, Artificial Sequence
  VARIANT 28: Xaa = Lys or Arg
HAEGTFTSDVSSYLEGQAAKEFIAWLVXGRX

SEQ ID NO: 15 - protein, 31 aa, Artificial Sequence
  VARIANT 2: Xaa = D-Ala
HXEGTFTSDVSSYLEGQAAKEFIAWLVXGRX

SEQ ID NO: 16 - protein, 39 aa, Artificial Sequence
  Description of Artificial Sequence synthetic peptide
HSDGTFTSDLSKQMEEEAVRLFIEWLKNGGPSSGAPPPS

SEQ ID NO: 17 - protein, 39 aa, Artificial Sequence
  Description of Artificial Sequence synthetic peptide
HGEGTFTSDLSKQMEEEAVRLFIEWLKNGGPSSGAPPPS

SEQ ID NO: 18 - protein, 31 aa, Artificial Sequence
  Description of Artificial Sequence synthetic peptide
HGEGTFTSDLSKQMEEAVRLFIEWLKNGGPY

SEQ ID NO: 19 - protein, 31 aa, Artificial Sequence
  Description of Artificial Sequence synthetic peptide
HGEGTFTSDLSKQMEEEAVRLFIEWLKNGGY

SEQ ID NO: 20 - protein, 31 aa, Artificial Sequence
  Description of Artificial Sequence synthetic peptide
DLSKQMEEEAVRLFIEWLKNGGPSSGAPPPS

SEQ ID NO: 21 - protein, 37 aa, Artificial Sequence
  Description of Artificial Sequence synthetic peptide
HDEFERHAEGTFTSDVSSYLEGQAAKEFIAWLVKGRK

SEQ ID NO: 22 - protein, 31 aa, Artificial Sequence
  Description of Artificial Sequence synthetic peptide
HAEGTFTSDVSSYLEGQAAKEFIAWLVKGRK

SEQ ID NO: 23 - protein, 40 aa, Artificial Sequence
  Description of Artificial Sequence synthetic peptide
HGEGTFTSDLSKQMEEEAVRLFIEWLKNGGPSSGAPPPSK

SEQ ID NO: 24 - protein, 40 aa, Artificial Sequence
  Description of Artificial Sequence synthetic peptide
HSDGTFTSDLSKQMEEEAVRLFIEWLKNGGPSSGAPPPSK

SEQ ID NO: 25 - protein, 31 aa, Artificial Sequence
  Description of Artificial Sequence synthetic peptide
HGEGTFTSDLSKEMEEEVRLFIEWLKNGGPY

SEQ ID NO: 26 - protein, 30 aa, Artificial Sequence
  Description of Artificial Sequence synthetic peptide
HGEGTFTSDLSKEMEEEVRLFIEWLKNGGY

SEQ ID NO: 27 - protein, 29 aa, Artificial Sequence
  Description of Artificial Sequence synthetic peptide
DLSKQMEEEAVRLFIEWLKGGPSSGPPPS

SEQ ID NO: 28 - protein, 30 aa, Artificial Sequence
  Description of Artificial Sequence synthetic peptide
XXDXXFXXXYXXXLXQLXAXXXLXXXXXXX

SEQ ID NO: 29 - protein, 15 aa, Artificial Sequence
  Description of Artificial Sequence synthetic peptide
QQGESNQERGARARL

SEQ ID NO: 30 - protein, 40 aa, Artificial Sequence
  BINDING 40: Xaa represents Lys (E-AEEA-MPA)-NH2 linked to Albumin Cysteine 34 thiol and where “E” represents Epsilon
HGEGTFTSDLSKQMEEEAVRLFIEWLKNGGPSSGAPPPSX

SEQ ID NO: 31 - protein, 31 aa, Artificial Sequence
  VARIANT 2: Xaa = D-Ala
HXEGTFTSDVSSYLEGQAAKEFIAWLVKGRX

SEQ ID NO: 32 - protein, 31 aa, Artificial Sequence
  Description of Artificial Sequence synthetic peptide
KXXDXXFXXXYXXXLXQLXAXXXLXXXXXXX

SEQ ID NO: 33 - protein, 45 aa, Artificial Sequence
  Description of Artificial Sequence synthetic peptide
XXDXXFXXXYXXXLXQLXAXXXLXXXXXXQQGESNQERGARARLK

SEQ ID NO: 34 - protein, 46 aa, Artificial Sequence
  Description of Artificial Sequence synthetic peptide
KXXDXXFXXXYXXXLXQLXAXXXLXXXXXXQQGESNQERGARARLK